SEQUENCE LISTING (11 sequences)

SEQ ID NO: 1 - DNA, 3822 bp, Artificial sequence
  synthetic sequence
atgtttgtgttcctggtgcttctgccacttgtttcctcacaatgcgtcaatctcacaacaagaacgcagcttccgcctgcatatacaaatagtttcaccagaggcgtttattatccagataaggtctttcgaagctctgtcctccattctacgcaagatctgttcttgccattcttttcaaacgtgacgtggtttcacgctattcacgtttctggcactaatggcacgaagcgctttgacaaccccgtccttccgttcaacgatggcgtctatttcgcgagtacagagaagtcaaatataatccgcggctggatatttggcacaaccctcgatagtaaaacacaatcccttctgatcgtaaataacgcaacgaatgtggtgattaaggtatgcgagtttcaattttgcaacgacccgttccttggcgtttattatcacaaaaataacaagtcctggatggagtccgaattcagagtgtattcttctgccaacaattgcacctttgagtacgtgtcccagcccttcttgatggatctggaaggaaaacagggcaatttcaagaatttgagagagttcgtcttcaaaaacatcgatggttattttaagatctatagcaagcacaccccgataaacctcgtacgggaccttccccaggggtttagcgcactggagcctcttgtagatctcccgattggcatcaatataaccaggtttcaaactcttcttgcacttcaccgaagctatcttacgcccggcgatagctcttccggttggacagccggcgccgcggcctactatgtgggatatctccagcctagaacctttctgcttaagtacaatgaaaacggcactattacagacgccgtagattgcgctctggatcctcttagcgaaactaagtgtactctgaaatcttttacggtagagaagggaatttatcaaacaagtaatttccgggtacaacccacagagagcatcgtccggtttccgaacattacgaatctttgcccgtttggcgaagtcttcaatgcaacacgcttcgcttccgtttatgcgtggaatcgcaagaggatctctaactgcgttgctgactacagtgttctttataacagcgctagcttttctacattcaaatgttacggggtaagccccaccaagctcaacgacttgtgttttaccaatgtttatgcggattctttcgtgattcgcggtgatgaagttaggcagattgcgccaggacagactgggaagattgccgactataactataagctccctgacgacttcactgggtgtgtcattgcttggaacagcaacaatctcgacagcaaagttggaggcaactataattatctctaccggctttttcgcaagtctaacctcaagcccttcgaaagagacatcagtactgagatctatcaagcaggaagcacgccatgcaacggtgtcgagggcttcaactgttattttcctcttcaatcctacggctttcaaccgacaaacggcgtcggatatcagccttatcgagtcgttgtactttctttcgaacttctgcacgcaccagctactgtctgcgggcctaaaaaatctacaaacctggtgaaaaataaatgcgttaattttaattttaacggtttgaccggcaccggggtccttactgagtcaaataaaaagttcctgccgtttcagcagtttggccgcgatattgccgatactacagacgcggtgcgggaccctcaaactctcgaaatcctggatataacgccttgctcttttggcggggtaagcgtcattaccccaggtacaaatacgtcaaatcaggtcgctgttctgtaccaagacgtcaattgcaccgaggtcccagtagctatccatgccgatcaactcacgccaacctggcgggtatactctacgggatccaacgtcttccaaaccagggcggggtgcctcatcggagccgaacacgtaaacaatagctatgagtgcgatattcccataggggccggaatctgcgcttcataccaaacccagactaattctccccgacgagcccgaagcgtggcatctcaaagcataatagcgtacacgatgagcttgggagcagaaaactcagtggcgtatagcaataactccatcgcgataccaaccaattttacaatcagtgtgactacagagatcttgccggttagtatgacaaagacttcagtggattgcacgatgtacatctgtggagatagtaccgagtgcagcaaccttttgttgcagtatggctcattttgtacccaactcaatcgggccctgaccggtattgcagttgaacaggataaaaacacacaagaggtctttgcacaagttaaacaaatatacaagaccccgcctatcaaagactttggtggttttaattttagccaaattcttcccgacccgtcaaagccgagtaagcggtcattcatagaggatctcctctttaataaggtaaccttggctgacgcgggatttattaaacagtacggtgactgtttgggagatattgcagctcgagacttgatctgcgcccagaaattcaacggtctgactgttttgcctccgctcttgacggacgagatgatcgcccagtatacttccgccttgctcgctggtacgatcacctccggctggacctttggcgctggagctgctctccagatcccattcgcgatgcagatggcttaccgatttaacggtattggcgttacacaaaatgtattgtacgagaaccaaaaactcatcgctaatcaattcaactcagctattggcaaaattcaagactcattgtcctctacagccagcgccttgggtaaattgcaagacgtagtgaaccaaaatgcacaggccttgaacactctggtcaagcaactcagttccaattttggtgcaatctcatccgttctcaacgatatactgtctcgcctggataaggtggaagctgaggtgcaaatcgacagactcataacgggaaggctgcagagtctgcagacctatgtcactcagcagcttattcgagcggccgaaatccgcgcttccgccaatttggctgctactaaaatgagcgaatgcgtactcgggcaatctaaacgagtcgacttttgtggcaaaggctaccacctcatgtcatttccccagagtgcgccacacggggtggttttccttcatgtcacctacgtgcccgcccaagaaaagaactttactaccgcaccggccatctgccatgatggaaaggcccatttcccgagagagggcgtatttgtctcaaacggtactcactggtttgtgacacaacgcaatttctacgaaccccaaattataaccactgacaacacatttgtctctggaaactgtgatgtcgttattggtatagttaacaacacggtctacgacccactccagccagagctggactcttttaaagaagaactcgacaagtattttaaaaatcacacgagtcccgacgttgaccttggagacattagtgggatcaacgcgagtgtcgttaacatccaaaaggagattgatcgcctcaatgaggtggccaagaatctcaacgaatccctcatagatctccaagagctcggtaagtatgagcagtatattaagtggccctggtatatatggctcggctttatcgccggcctgattgcaatagttatggttacgataatgctgtgctgtatgacttcatgctgttcttgtttgaaaggctgttgctcatgtgggagttgttgcaagtttgatgaggacgacagtgaacccgtattgaagggtgtgaagcttcattacacttaa

SEQ ID NO: 2 - protein, 1273 aa, Artificial sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3627 bp, Artificial sequence
  synthetic sequence
atgtttgtgttcctggtgcttctgccacttgtttcctcacaatgcgtcaatctcacaacaagaacgcagcttccgcctgcatatacaaatagtttcaccagaggcgtttattatccagataaggtctttcgaagctctgtcctccattctacgcaagatctgttcttgccattcttttcaaacgtgacgtggtttcacgctattcacgtttctggcactaatggcacgaagcgctttgacaaccccgtccttccgttcaacgatggcgtctatttcgcgagtacagagaagtcaaatataatccgcggctggatatttggcacaaccctcgatagtaaaacacaatcccttctgatcgtaaataacgcaacgaatgtggtgattaaggtatgcgagtttcaattttgcaacgacccgttccttggcgtttattatcacaaaaataacaagtcctggatggagtccgaattcagagtgtattcttctgccaacaattgcacctttgagtacgtgtcccagcccttcttgatggatctggaaggaaaacagggcaatttcaagaatttgagagagttcgtcttcaaaaacatcgatggttattttaagatctatagcaagcacaccccgataaacctcgtacgggaccttccccaggggtttagcgcactggagcctcttgtagatctcccgattggcatcaatataaccaggtttcaaactcttcttgcacttcaccgaagctatcttacgcccggcgatagctcttccggttggacagccggcgccgcggcctactatgtgggatatctccagcctagaacctttctgcttaagtacaatgaaaacggcactattacagacgccgtagattgcgctctggatcctcttagcgaaactaagtgtactctgaaatcttttacggtagagaagggaatttatcaaacaagtaatttccgggtacaacccacagagagcatcgtccggtttccgaacattacgaatctttgcccgtttggcgaagtcttcaatgcaacacgcttcgcttccgtttatgcgtggaatcgcaagaggatctctaactgcgttgctgactacagtgttctttataacagcgctagcttttctacattcaaatgttacggggtaagccccaccaagctcaacgacttgtgttttaccaatgtttatgcggattctttcgtgattcgcggtgatgaagttaggcagattgcgccaggacagactgggaagattgccgactataactataagctccctgacgacttcactgggtgtgtcattgcttggaacagcaacaatctcgacagcaaagttggaggcaactataattatctctaccggctttttcgcaagtctaacctcaagcccttcgaaagagacatcagtactgagatctatcaagcaggaagcacgccatgcaacggtgtcgagggcttcaactgttattttcctcttcaatcctacggctttcaaccgacaaacggcgtcggatatcagccttatcgagtcgttgtactttctttcgaacttctgcacgcaccagctactgtctgcgggcctaaaaaatctacaaacctggtgaaaaataaatgcgttaattttaattttaacggtttgaccggcaccggggtccttactgagtcaaataaaaagttcctgccgtttcagcagtttggccgcgatattgccgatactacagacgcggtgcgggaccctcaaactctcgaaatcctggatataacgccttgctcttttggcggggtaagcgtcattaccccaggtacaaatacgtcaaatcaggtcgctgttctgtaccaagacgtcaattgcaccgaggtcccagtagctatccatgccgatcaactcacgccaacctggcgggtatactctacgggatccaacgtcttccaaaccagggcggggtgcctcatcggagccgaacacgtaaacaatagctatgagtgcgatattcccataggggccggaatctgcgcttcataccaaacccagactaattctccccgacgagcccgaagcgtggcatctcaaagcataatagcgtacacgatgagcttgggagcagaaaactcagtggcgtatagcaataactccatcgcgataccaaccaattttacaatcagtgtgactacagagatcttgccggttagtatgacaaagacttcagtggattgcacgatgtacatctgtggagatagtaccgagtgcagcaaccttttgttgcagtatggctcattttgtacccaactcaatcgggccctgaccggtattgcagttgaacaggataaaaacacacaagaggtctttgcacaagttaaacaaatatacaagaccccgcctatcaaagactttggtggttttaattttagccaaattcttcccgacccgtcaaagccgagtaagcggtcattcatagaggatctcctctttaataaggtaaccttggctgacgcgggatttattaaacagtacggtgactgtttgggagatattgcagctcgagacttgatctgcgcccagaaattcaacggtctgactgttttgcctccgctcttgacggacgagatgatcgcccagtatacttccgccttgctcgctggtacgatcacctccggctggacctttggcgctggagctgctctccagatcccattcgcgatgcagatggcttaccgatttaacggtattggcgttacacaaaatgtattgtacgagaaccaaaaactcatcgctaatcaattcaactcagctattggcaaaattcaagactcattgtcctctacagccagcgccttgggtaaattgcaagacgtagtgaaccaaaatgcacaggccttgaacactctggtcaagcaactcagttccaattttggtgcaatctcatccgttctcaacgatatactgtctcgcctggataaggtggaagctgaggtgcaaatcgacagactcataacgggaaggctgcagagtctgcagacctatgtcactcagcagcttattcgagcggccgaaatccgcgcttccgccaatttggctgctactaaaatgagcgaatgcgtactcgggcaatctaaacgagtcgacttttgtggcaaaggctaccacctcatgtcatttccccagagtgcgccacacggggtggttttccttcatgtcacctacgtgcccgcccaagaaaagaactttactaccgcaccggccatctgccatgatggaaaggcccatttcccgagagagggcgtatttgtctcaaacggtactcactggtttgtgacacaacgcaatttctacgaaccccaaattataaccactgacaacacatttgtctctggaaactgtgatgtcgttattggtatagttaacaacacggtctacgacccactccagccagagctggactcttttaaagaagaactcgacaagtattttaaaaatcacacgagtcccgacgttgaccttggagacattagtgggatcaacgcgagtgtcgttaacatccaaaaggagattgatcgcctcaatgaggtggccaagaatctcaacgaatccctcatagatctccaagagctcggtaagtatgagcagtaa

SEQ ID NO: 4 - protein, 1276 aa, Artificial sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYDYPKYSEESKLNREKIDGVKLESMGVYQILAIYSTVASSLVLLVSLGAISFWMCSNGSLQCRICI

SEQ ID NO: 5 - protein, 13 aa, Artificial sequence
  synthetic sequence
MFVFLVLLPLVSS

SEQ ID NO: 6 - protein, 305 aa, Artificial sequence
  synthetic sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNF

SEQ ID NO: 7 - protein, 192 aa, Artificial sequence
  synthetic sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 8 - protein, 698 aa, Artificial sequence
  synthetic sequence
VVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 9 - protein, 30 aa, Artificial sequence
  synthetic sequence
GTYDYPKYSEESKLNREKIDGVKLESMGVY

SEQ ID NO: 10 - protein, 26 aa, Artificial sequence
  synthetic sequence
QILAIYSTVASSLVLLVSLGAISFWM

SEQ ID NO: 11 - protein, 12 aa, Artificial sequence
  synthetic sequence
CSNGSLQCRICI

(19) United States
(12) Patent Application Publication
(10) Pub. No.: US 2024/0269265 A1
(43) Pub. Date: Aug. 15, 2024

(54) Peptide VLP-Based Vaccines

(71) Applicant: CHIMERON BIO CORPORATION, Philadelphia, PA (US)

(72) Inventors: Jolly Mazumdar Chendrimada, Philadelphia, PA (US); Thimmaiah Ponnappa Chendrimada, Philadelphia, PA (US)

(21) Appl. No.: 18/567,621

(22) PCT Filed: Jun. 9, 2022
(86) PCT No.: PCT/US2022/032861

§ 371 Date: Dec. 6, 2023

Related U.S. Application Data
(60) Provisional application No. 63/208,895, filed on Jun. 9, 2021.

(51) Int. Cl. A61K 39/215; A61K 39/00; A61K 39/145; A61K 39/205; A61K 39/21; A61P 31/14; C12N 7/00

(52) U.S. Cl. CPC A61K 39/215; A61K 39/145; A61K 39/205; A61K 39/21; A61P 31/14; C12N 7/00; A61K 2039/5258; A61K 2039/575; C12N 2770/20023; C12N 2770/20034; C12N 2770/20052; C12N 2770/20071

(57) ABSTRACT

Provided herein are peptide VLP vaccines that deliver at least one immunogenic antigen to a subject and induce a protective immune response in the subject. Additionally, provided are related methods and compositions.

### CROSS-REFERENCE TO RELATED APPLICATIONS

This application claims the benefit of and priority to U.S. Provisional Application No. 63/208,895 filed Jun. 9, 2021, which is incorporated by reference in its entirety.

### SEQUENCE LISTING

The instant application contains a Sequence Listing with 11 sequences, which has been submitted via EFS-Web and is hereby incorporated herein by reference in its entirety. Said ASCII copy, created on Jun. 9, 2022, is named 46556_SEQlisting_.txt, and is 51 kilobytes (KB) in size.

### 1. BACKGROUND

Globalization has an effect on both the transmission of pathogens and the availability of a pool of susceptible individuals. Global travel means that pathogens can travel around the world more easily. It also makes the pool of susceptible people larger. As a consequence, any new infectious disease, arising for example by mutation of an animal pathogen bacterial or viral (e.g. MERS, SARS, bird flu, Ebola, HIV etc.), has the opportunity to spread to susceptible individuals, and to be maintained within the human population for the long term.

An example of such a disease is the SARS-COV-2, a recently emerged highly pathogenic human coronavirus. This disease has been declared a pandemic by the World Health Organization (WHO) and is having severe effects on both individual lives and economies around the world. Infection with SARS-COV-2 is characterized by a broad spectrum of clinical syndromes, which range from asymptomatic disease or mild influenza-like symptoms to severe pneumonia and acute respiratory distress syndrome

In view of the high morbidity and mortality that can follow infection with bacterial and viral diseases, especially emerging pathogens, there is an urgent need for preventive vaccines.

### 2. SUMMARY

Described herein are compositions relating to virus-like particles (VLPs) and methods for making and using the described VLPs. In certain embodiments, the compositions provide delivery of at least one immunogenic antigen of an infectious agent as a heterologous peptide attached to a VLP shell (e.g, a “decorated” peptide-VLP). In additional embodiments, the compositions include SARS-COV-2 peptide VLPs. In additional embodiments, the compositions include plasmids and cells used to produce the described peptide VLPs.

Provided herein is a virus like particle (VLP), the VLP comprising:
a. a capsid protein comprising a retroviral gag protein, and
b. an envelope protein, comprising at least one antigenic heterologous polypeptide or fragment thereof, wherein the VLP does not contain alphavirus genetic material.

In certain embodiments, the infectious agent is a virus. In certain embodiments, the virus is a coronavirus. In certain embodiments, the coronavirus is SARS-COV-2. In certain embodiments, the immunogenic antigen is a surface antigen. In certain embodiments, the surface antigen has 80% identity to a coronavirus spike protein, a membrane protein, a hemagglutinin esterase (HE), a hemagglutinin from influenza virus, or an envelope protein, or any fragment or combination thereof.

In certain embodiments, the surface antigen has 80% identity to a coronavirus spike protein or a chimeric protein comprising the SARS-COV-2 ectodomain spike glycoprotein (S-protein) and hemagglutinin (HA) transmembrane region from Influenza virus. In certain embodiments, the spike protein sequence shares at least 90% identity with a coronavirus spike protein of SEQ ID NO:2 or ectodomain amino acids 1-1208 of SEQ ID NO: 2, or a chimeric protein comprising the SARS-COV-2 ectodomain spike glycoprotein (S-protein) and hemagglutinin (HA) transmembrane region from Influenza virus (SEQ ID NO:4-11) or a combination thereof.

In certain embodiments, the spike protein sequence comprises SEQ ID NO: 2 or the ectodomain region of amino acids 1-1208 of SEQ ID NO: 2, or comprises a chimeric protein of the SARS-COV-2 ectodomain spike glycoprotein (S-protein) and hemagglutinin (HA) transmembrane region from Influenza virus (SEQ ID NO:4-11), or a combination thereof.

In certain embodiments, the spike protein sequence is encoded by a codon optimized sequence of SEQ ID NO: 1; or a codon optimized ectodomain sequence of amino acids 1-1208 of SEQ ID NO: 1, or a codon optimized sequence of SEQ ID NO:3 encoding the ectodomain COVID-19 spike glycoprotein (S-protein) with hemagglutinin (HA) transmembrane region from Influenza.

In certain embodiments, the VLP does not comprise or express a retroviral pol gene.

In certain embodiments, the retroviral gag protein is encoded by a polynucleotide sequence derived from Rous sarcoma virus (RSV).

In certain embodiments, the VLP is capable of presenting the immunogenic antigen to a target cell in a subject, after which the subject is capable of mounting a cellular and/or humoral immune response to SARS-COV-2 in the subject.

In certain embodiments, the immune response including a T cell response is induced by a dosing regimen comprising one or two administrations of the VLP, wherein the dose is sufficient to induce an immune response against coronavirus in a subject.

In certain embodiments, the dosing regimen comprises one administration of the VLP, wherein the one dose administration is sufficient to induce an immune response against the coronavirus in a subject.

In certain embodiments, provided herein is a pharmaceutical composition comprising the VLP of any of the previous embodiments. In certain embodiments, the composition comprises, optionally, a pharmaceutically acceptable carrier, diluent, adjuvant and/or additive, or any combination thereof.

In certain embodiments, the composition is capable of inducing an immune response against coronavirus in a subject.

In certain embodiments, following administration of the composition to the subject, the VLP is capable of inducing a T cell response against coronavirus. In certain embodiments, the coronavirus is SARS-COV-2 (COVID-19).

In certain embodiments, the composition is for use in diminishing or preventing a coronavirus infection in a mammalian subject. In certain embodiments, said diminishing or preventing comprises inducing coronavirus-specific immunity against SARS-COV-2 (COVID-19)

In certain embodiments, the composition is for use in inducing cellular and or humoral immunity in a mammalian subject. In certain embodiments, the composition is for inducing or eliciting an immune response in a mammalian subject. In certain embodiments, the composition is for said inducing or eliciting an immune response is an immune response against SARS-COV-2 (COVID-19). In certain embodiments the composition is for use in inducing neutralizing antibodies against SARS-COV-2 in a mammalian subject.

Further provided herein is a method of diminishing or preventing a coronavirus infection in a mammalian subject comprising administering the VLP or pharmaceutical composition described herein to the subject.

Further provided herein is a method of inducing cellular and or humoral immunity against a coronavirus in a mammalian subject, comprising administering the VLP or pharmaceutical composition described herein to the subject.

Further provided herein is a method of eliciting an immune response against a coronavirus in a mammalian subject, comprising administering the VLP or pharmaceutical composition described herein to the subject.

Further provided herein is a method of inducing neutralizing antibodies against SARS-CoV-2 in a subject, comprising administering the VLP or pharmaceutical composition described herein to the subject. In certain embodiments, the method further includes inducing a T cell response against the coronavirus. In certain embodiments, the said T cell response is induced by a regimen comprising one or at least two administrations. In certain embodiments, the immune response is induced by a regimen comprising one administration of the VLP or pharmaceutical composition. In certain embodiments, the coronavirus is SARS-COV-2. In certain embodiments, the VLP is capable of delivering the immunogenic antigen to a target cell in a subject, after which the subject is capable of mounting a cellular and/or humoral immune response to SARS-COV-2 in the subject.

Further provided herein is method of producing the VLP described herein, comprising: co-transforming a eukaryotic cell with:
i. a first plasmid or vector comprising a polynucleotide encoding the retroviral gag protein; and
ii. a second plasmid or vector comprising a polynucleotide encoding an envelope protein comprising one or more heterologous polypeptides or fragment thereof or encoding one or more polypeptide or fragment thereof which exhibits at least 80% identity to at least one immunogenic antigen of an infectious agent;
iii. culturing the co-transformed eukaryotic cell under conditions suitable to cause each vector to produce its encoded product, thereby producing the VLP; and
iv. isolating the VLP from the eukaryotic cell.

A VLP produced by any of the methods described above.

Further provided herein is method of diminishing or preventing a coronavirus infection in a mammalian subject comprising administering the VLP described herein to a subject.

Further provided herein is method for inducing cellular and or humoral immunity in a mammalian subject, comprising administering the VLP described herein to a subject.

Further provided herein is method of eliciting an immune response in a mammalian subject, comprising administering the VLP described herein to a mammalian subject.

Further provided herein is method of inducing neutralizing antibodies against SARS-CoV-2 in a mammalian subject, comprising administering the VLP described herein to a subject.

Further provided herein is method, wherein the method further includes inducing a T cell response against the coronavirus. In certain embodiments, the said T cell response is induced by a regimen comprising at least one or at least two administrations. In certain embodiments, the coronavirus is SARS-COV-2 (COVID-19). In certain embodiments, the VLP is capable of delivering the immunogenic antigen to a target cell in the subject, after which the subject is capable of mounting a cellular and/or humoral immune response to SARS-COV-2 in the subject.

In certain embodiments, the VLP is stable at from the range of about 4° C.-10° C. for at least about one-six months. In certain embodiments, the VLP is stable for at least about six to nine months. In certain embodiments, the VLP is stable for at least about nine to twelve months.

In certain embodiments, the VLP is stable at about -80° C. for at least about one year. In certain embodiments, the VLP is stable for about two years. In certain embodiments, the VLP is stable for about three years. In certain embodiments, the dose of the VLP is equivalent to about 1 μg-1000 μg of total protein. In certain embodiments, the VLP is capable of delivering one or more immunogenic antigens to a target cell in a subject exposing the subject to the antigen, after which exposure the subject is capable of producing a cellular and/or humoral immune response to Severe Acute Respiratory Syndrome (SARS-COV), SARS-COV-2, and variants of SARS-COV-2.

### 3. BRIEF DESCRIPTION OF THE DRAWINGS

These and other features, aspects, and advantages of the present invention become better understood with regard to the following description, and accompanying drawings, where:

FIG. 1A: Delivery of immunogenic antigen by a decorated a chimeric peptide VLP. The viral-like protein (VLP) delivery particle expresses the VSV-G protein on its surface, that will target, in general, to immune cells. The peptide VLP's deliver one or more immunogenic antigens to a subject, and this exposure/ delivery provides exposure to the one or more immunogenic antigens producing or inducing an immune response, including a T cell response, against coronavirus in the subject.

FIG. 1B: Design of the peptide SARS-COV-2 VLP vaccine. The VLP is typically generated by utilizing two separate plasmids, one encoding an envelope protein which comprises either full-length spike protein (CB-108) or the ectodomain of SARS-COV-2 spike glycoprotein fused with the HA domain, a linker, and the HA cytoplasmic tail (CB-109), and a second plasmid containing a capsid protein, which in this instance is an RSV-GAG protein.

FIG. 2A: Nucleotide sequence of the full length Covid-19 spike glycoprotein (S-protein) (SEQ ID NO:1). Genbank ID NC_045512 REGION: 21563 . . . 25384.

FIG. 2B: Amino acid sequence of the full length Covid-19 spike glycoprotein (S-protein) (SEQ ID NO:2). Genbank ID 43740568. Underlined is the S1 domain.

FIG. 2C shows the polynucleotide sequence (codon optimized) of the ectodomain COVID-19 spike glycoprotein (S-protein) with hemagglutinin (HA) transmembrane region from Influenza (SEQ ID NO:3). FIGS. 2D-2F show the amino acid sequence of the ectodomain COVID-19 spike glycoprotein (S-protein) fused with the hemagglutinin (HA) transmembrane region from influenza, which serves as an anchor in the VLP gag shell, along with the other amino acid domain sequences (SEQ ID NO:4-11).

FIG. 3: Confirmation of Particle composition of CB-109. The western blot illustrates Spike and GAG protein expression on the CB-109 VLP particle.

FIG. 4. Study design. Peptide SARS-COV-2 VLP vaccine candidates were tested in young naïve female Balb/c mice in a Prime and Boost design. The peptide SARS-COV-2 VLP vaccine was tested at multiple dose levels. Both vaccine candidates are active and were well tolerated. Effective neutralizing antibody production was observed in a prime and boost setting.

FIGS. 5A-5B are graphs showing the generation of neutralizing antibodies against SARS-COV-2 by mice following vaccination with SARS-COV-2 peptide VLP candidates.

FIGS. 6A-6C are graphs illustrating the Th1/Th2 skew in response to SARS-COV-2 VLP-peptide vaccine. IgG data are shown, specifically IgG1 (FIG. 6A) and IgG2a (FIG. 6B) responses in mice vaccinated with VLP-peptide. N=5 animals. The mice demonstrated a Th1 bias (FIG. 6C) Data shown as Mean±SD. Significance compared to antigen control was determined by performing a one-way ANOVA; **p<0.001, ***p<0.0001.

FIG. 7 is a graph illustrating temperature stability of the SARS-COV-2 peptide VLP vaccine at 4° C. over the course of twelve weeks, measuring the presence of the spike protein.

FIGS. 8A-8B illustrate safety tox studies in rabbits. A human equivalent efficacious dose of the peptide SARS-COV-2 VLP was tested in rabbits using a prime+boost design. There was no impact on body weight gain, food consumption (FIG. 8A) and other parameters measured (FIG. 8B).

### 4. DETAILED DESCRIPTION

### 4.1. Peptide VLPs Eliciting an Immune Response Against or Preventing Viral-Mediated Infection and Related Conditions, including against Coronaviruses

While not wishing to be bound by theory, the present disclosure is based at least in part on the ability of a peptide VLP vaccine (e.g. the VLP vaccine) to deliver one or more immunogenic antigens to the patient, and subsequently elicit an immune response in the patient, which immune response is sufficient to prevent or significantly decrease the duration of an infection by an infectious agent, such as SARS-COV-2, as described herein below.

As described further herein, embodiments relate to peptide VLP some of which are decorated on their surface with an antigenic polypeptide, such as the coronavirus full length spike protein. In this instance, the coronavirus spike protein has its own transmembrane (TM) domain, and once the protein is formed in the production cell, it will bind to the GAG protein and form a peptide VLP.

In additional embodiments, the VLP comprises a capsid and an “envelope” surface antigenic spike polypeptide in which the native TM and cytoplasmic tail to the spike protein have been removed and replaced with a “heterologous” transmembrane domain and cytoplasmic tail from influenza hemagglutinin (HA). This HA TM domain binds to the capsid, e.g. GAG for form the VLP particle. In some embodiments the HA TM domain anchored to the heterologous antigenic polypeptide forms a more stable peptide VLP.

### 4.2. Definitions

Unless defined otherwise, all technical and scientific terms used herein have the meaning commonly understood by one of ordinary skill in the art to which the invention pertains.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms.

As used herein “preventing” a disease refers to inhibiting the full development of a disease.

The term “biological sample” refers to any tissue, cell, fluid, or other material derived from an organism (e.g., human subject). In certain embodiments, the biological sample is serum or blood.

“Antibody” as used herein encompasses polyclonal and monoclonal antibodies and refers to immunoglobulin molecules of classes IgA (e.g., IgA1 or IgA2), IgD, IgE, IgG (e.g., IgG1, IgG2, IgG3 and IgG4) or IgM, or fragments, or derivatives thereof, including without limitation Fab, F(ab′)2, Fd, single chain antibodies, diabodies, bispecific antibodies, bifunctional antibodies, humanized antibodies, and various derivatives thereof.

As used herein, the term “neutralizing antibody” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

As used herein in connection with a viral infection and vaccination, the terms “protective immune response” or “protective immunity” refer to an immune response that that confers some benefit to the subject in that it prevents or reduces the infection or prevents or reduces the development of a disease associated with the infection. As an example, the presence of SARS-COV-2 neutralizing antibodies in a subject can indicate the presence of a protective immune response in the subject.

“Virus-like particle” (VLP), as used herein, refers to a structure resembling a virus particle. In preferred embodiments, a VLP contains at least one heterologous polypeptide or fragment thereof displayed on the surface of the particle. A virus-like particle as provided herein lacks all or part of the replicative and non-replicative components of the viral genome, i.e. does not contain any alphavirus structural and non-structural proteins and furthermore, contains no genetic material.

The terms peptide VLP vaccine, “VLP vaccine composition”, or “VLP vaccine”, which are used herein interchangeably, refer to a peptide VLP composition comprising at least one heterologous polypeptide or fragment thereof, which is present on the surface of the VLP producing a peptide VLP vaccine that is capable of inducing an immune response in a subject (e.g., humoral and/or cellular response). In certain embodiments, the immune response is a protective immune response. A vaccine may be administered for the prevention or treatment of a disease, such as an infectious disease.

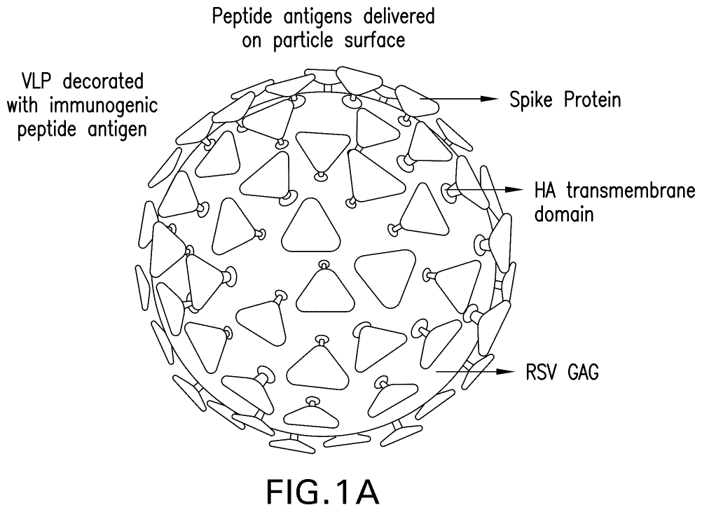

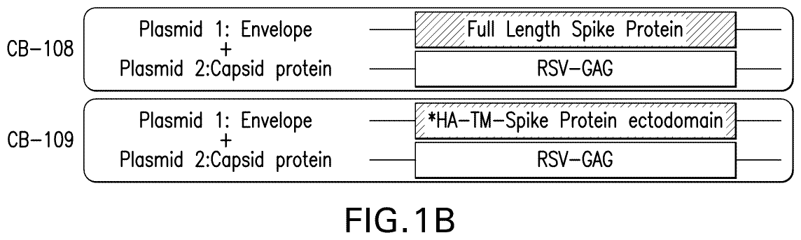

The term envelope, as used herein refers to the at least one antigenic heterologous polypeptide or fragment thereof which binds to the capsid protein of the VLP and is surface expressed and assembled on the VLP, and in certain embodiments may include one or more linkers, C-terminal transmembrane anchor domains, and one or more cytoplasmic tail regions, which serve to anchor the polypeptide in the capsid for surface display (See FIG. 1A). Such a surface envelope protein as used herein occurs as a membrane anchored “peplomer” which is known in the art as an external polypeptide on the surface of the VLP and is bound/attached to the capsid (e.g. GAG), which forms the peptide decorated VLP, as described herein.

The term “fusogen” or “fusogenic molecule” is used herein to refer to any molecule that can trigger membrane fusion when present on the surface of a virus particle. A fusogen can be, for example, a protein (e.g., a viral glycoprotein) or a fragment or derivative thereof.

The term “replication-competent” is used herein to refer to viruses (including wild-type and recombinant viral particles) that are capable of infecting and propagating within a susceptible cell.

The term “replication incompetent” is used herein to refer to VLPs that are not capable of propagating within a susceptible or target cell. Such a VLP can bind to, fuse and transfer its nucleic acid payload into the target cell, but has no viral replication components or structural proteins to assemble or be capable of viral replication.

The term “encoding” can refer to encoding from either the (+) or (−) sense strand of the polynucleotide for expression in the virus particle.

Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (on the world wide web at: ncbi.nlm.nih.gov/).

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme-PAM (Percent Accepted Mutations) 0, k-tuple=2, Mismatch Penalty=1, Joining Penalty-20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The term “effective” applied to dose or amount refers to that quantity of a compound (e.g., a recombinant VLP) or composition (e.g., pharmaceutical, vaccine or immunogenic and/or antigenic composition) that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

As used herein, the phrase “a subject in need thereof” means a human or non-human animal that exhibits one or more symptoms or indicia of a disease or disorder associated with a coronavirus infection, and/or who is at risk of developing a disease or disorder associated with an infection. In certain embodiments, the coronavirus is SARS-COV-2. In certain embodiments, the disease or disorder is COVID-19. In certain embodiments, the COVID-19 disease symptoms include, but are not limited to, fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock and death in severe cases.

The terms “individual” or “subject” or “patient” or “animal” refers to humans, veterinary animals (e.g., cats, dogs, cows, horses, sheep, pigs, etc.) and experimental animal models of diseases (e.g., mice, rats, ferrets, monkeys, etc.). In a preferred embodiment, the subject is a human.

The terms “nucleic acid”, “polynucleotide” and “nucleotide” are used interchangeably and encompass both DNA and RNA, including positive- and negative-stranded, single- and double-stranded, unless specified otherwise.

The phrase “pharmaceutically acceptable”, as used in connection with compositions described herein, refers to molecular entities and other ingredients of such compositions that are physiologically tolerable and do not typically produce untoward reactions when administered to a subject (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “Coronavirus” as used herein refers to the subfamily Coronavirinae within the family Coronaviridae, within the order Nidovirales. Based on the phylogenetic relationships and genomic structures, this subfamily consists of four genera: Alphacoronavirus, Betacoronavirus, Gammacoronavirus and Deltacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-COV-2, SARS-COV and MERS-COV, which cause severe respiratory syndrome in humans. The other four human coronaviruses, HCoV-NL63, HCoV-229E, HCoV-OC43 and HKU1, induce only mild upper respiratory diseases in immunocompetent hosts, although some of them can cause severe infections in infants, young children and elderly individuals. Additional non-limiting examples of commercially important coronaviruses include transmissible gastroenteritis coronavirus (TGEV), porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus. Reviewed in Cui et al., Nature Reviews Microbiology, 2019, 17:181-192; Fung et al., Annu. Rev. Microbiol., 2019, 73:529-557.

Severe acute respiratory syndrome beta coronavirus 2 and SARS-COV-2 (COVID-19) are used interchangeably herein. In certain embodiments, the virus is SARS-COV-2, also referred to as nCOV-2, nCoV2 or 2019-nCOV. The terms “nCoV2”, “nCOV-2”, “SARS-COV-2” and “SARS-COV-2 (COVID-19)” are used interchangeably herein. In particular embodiments, the peptide VLP vaccine prevents severe acute respiratory syndrome (SARS) in the patient/subject.

### 4.3. Peptide VLPs Comprising at Least One Immunogenic Antigen of an Infectious Agent

Aspects provided herein include a virus like particle (VLP), the VLP comprising:
a. a retroviral gag protein,
b. a envelope protein comprising at least one antigenic heterologous polypeptide or fragment thereof or polypeptide or fragment thereof which exhibits at least 80% identity to at least one immunogenic antigen of an infectious agent,

wherein the VLP does not contain an alphavirus genetic material.

In particular embodiments, the peptide-VLP vaccine provides the subject with an immune response (e.g. induces production of neutralizing antibodies in the patient/subject) that prevents or lessens coronavirus disease 2019 (COVID-19).

### Peptide VLPs

The peptide VLPs described herein are self-assembled nanoparticles. In certain embodiments, the peptide VLPs are pseudotyped with at least one antigenic peptide which can be an antigenic component of an infectious agent. In certain embodiments, the peptide VLPs generally have the following features they comprise at least one antigenic component from an infectious agent on their surface; they will have a viral core comprising a retroviral gag protein from any member of the Retroviridae family, or, in some embodiments, a gag fusion protein; they will have a surface protein to facilitate peptide presentation to an antigen presenting cell (APC), and they will not contain alphavirus structural proteins, and will be replication incompetent.

As used herein, the envelope of the VLP refers to the surface protein which is attached to the surface of the VLP (a heterologous polypeptide or fragment thereof), forming the decorated peptide VLP and is anchored to the capsid by a transmembrane domain and in some instances a linker and a optionally a suitable cytoplasmic tail.

The VLPs described herein will be useful as a vaccine for contacting a target cell, such as an APC with at least one immunogenic peptide such as an antigenic component of an infectious agent. Accordingly, the described VLPs may express on their surface at least one antigenic component of an infectious agent.

The described VLPs may also comprise a viral gag protein to provide a viral core structure to the particle. The gag protein is the core structural protein of retroviruses and, in some instances, is capable of forming enveloped virus cores when expressed in eukaryotic cells. This property makes gag proteins particularly useful in the production of VLPs, because they can form the basic structural aspect of the particle and allow for packaging of RNA associated with a retroviral packaging signal sequence. Those skilled in the relevant art will understand that a gag protein from any retrovirus (i.e. any member of the family Retroviridae) may be used to produce the plasmids and VLPs described herein (See, for example exemplary methods described in WO2013/148302 and WO 2015/095167). In some embodiments the gag protein may be derived from Rous sarcoma virus. In some embodiments the gag protein may be derived from murine leukemia virus. In alternative embodiments the gag protein may be derived from SIV, HIV, human T-lymphotropic virus, or similar retrovirus.

Another component of the VLPs described herein is a protein that is pseudotyped to the VLP envelope . A class of proteins suitable for this purpose is viral fusion proteins, which facilitate virus infection of cells by allowing an enveloped virus to fuse its membrane with that of a host cell. Many of viral fusion proteins also have known, or suspected, cellular receptor proteins that may allow for targeting of selected cell types, or in cases of more ubiquitous receptors, such as sialic acid for influenza virus, more generalized targeting may be achieved. In some instances, the antigenic polypeptide or protein, can include any portion or fragment of a viral attachment protein, ligands of cellular receptors, receptors of cellular ligands, or accessory proteins, thus proteins of this sort may also be present on the VLP surface to form the peptide VLP. Alternatively, in some embodiments the described VLPs may have a viral attachment protein of another virus, a ligand of a cellular receptor, a receptor of a cellular ligand, or an accessory protein to facilitate presentation of the peptide VLP antigen to an APC, for example. Similarly, the described VLPs may be produced to have more than one surface protein in the VLP envelope, as this may facilitate fusion to a select variety of cell types. In some embodiments the VLP surface protein(s) will be a type-I membrane protein, which will allow the extracellular domain of the protein to be oriented extracellularly when present on the cell surface. This will also allow the surface protein to be correctly oriented following budding of a VLP from a packaging cell. Expression of such proteins in a cell will typically result in the cell surface being coated with the proteins, so that budding of a VLP from any part of the cell membrane will provide the VLP with some amount of the fusion protein on its surface. In certain embodiments, the envelope protein can be selected from any of the following: hemagglutinin (such as hemagglutinin from influenza virus) , neuraminidase, Rous sarcoma virus (RSV) fusion protein, an E protein of tick borne encephalitis virus and dengue fever virus, the El protein of SFV, baculovirus gp64, and Vesicular stomatitis (Indiana) virus-G (VSV-G) protein, preferably a glycoprotein, or fragment or derivative thereof, more preferably from a RNA virus or a retrovirus, or fragment or derivative thereof, most preferably VSV-G or EnvA, or an alteration of VSV-G. The VLP described herein may be capable of binding to a eukaryotic target cell, preferably a human cell. The binding of the VLP may be specific to a target cell. In some embodiments the VLP described herein is not cytopathic to the target cell.

In some embodiments the VLPs described herein may include a transmembrane region from a vesicular stomatitis virus G protein (VSV-G) to mediate attachment to the GAG protein of the VLP. In some embodiments the VLPs described herein include an influenza hemagglutinin transmembrane fragment to mediate attachment of the heterologous polypeptide to the capsid.

In certain embodiments, a heterologous polypeptide or fragment thereof can include an antigen or antigenic polypeptide. As used herein, “antigen” refers typically to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide (e.g. polypeptide) or protein, or fragment which is capable of being presented to T-cells.

The VLPs described herein may comprise an envelope protein which comprises at least one immunogenic antigen of an infectious agent and a capsid protein comprising a retroviral gag protein; wherein the VLP does not contain an alphavirus structural protein or any alphavirus genetic material. Based on this description, those skilled in the art will readily understand that the described VLPs may be modified to incorporate aspects of viruses that may facilitate VLP stability. Such modifications should be understood to be within the scope of the disclosures provided herein.

In addition to the capsid protein, the peptide VLP is comprised of an envelope protein—which is in this instance comprises the heterologous polypeptide or fragment thereof anchored or bound to the capsid by at least a transmembrane domain (TM). In certain embodiments, this transmembrane domain can be a hemagglutinin transmembrane region, such as from influenza. The hemagglutinin TM binds especially well to the RSV GAG capsid. In certain embodiments, the transmembrane domain is from an RSV or any suitable transmembrane domain with a similar function to anchor the heterologous polypeptide to the capsid. The transmembrane region portion of the antigen is used to anchor the heterologous polypeptide or protein on the GAG surface, i.e. the VLP capsid surface, as shown in FIG. 1A. In certain embodiments a linker is also used to link the antigenic heterologous polypeptide or fragment thereof to the TM domain. In additional embodiments, a cytoplasmic tail of the transmembrane domain is also included in the envelope construct. In certain embodiments, such as for CB-108, the full-length Coronavirus spike protein has a transmembrane domain, which serves to anchor the protein to the capsid, i.e., the RSV-GAG. These envelope construct components can be mixed and matched to achieve optimum assembly of the antigenic heterologous polypeptide or fragment thereof to the capsid to form the peptide VLP particle. A schematic of exemplary designs of the peptide VLP elements are shown in FIGS. 1A-B. The peptide VLP is self-assembling, because the heterologous polypeptide or antigen once expressed in the production cell line binds to the GAG protein and makes the peptide VLP. The VLP particle is excreted into the media and purified to produce the peptide-VLP vaccine product.

In some embodiments the VLPs described herein include on their surface the amino acid sequence of the ectodomain COVID-19 spike glycoprotein (S-protein) with hemagglutinin (HA) transmembrane region from Influenza (SEQ ID NO:4-11). In some embodiments the VLPs described herein include an amino acid sequence with 80%, 85%, 90%, or 95% identity to the ectodomain COVID-19 spike glycoprotein (S-protein) fused with the hemagglutinin (HA) transmembrane region from Influenza (SEQ ID NO:4-11).

In certain embodiments, the VLP does not comprise or express a retroviral pol gene.

In certain embodiments, the gag protein is any gag protein from the family Retroviridae.

In certain embodiments, the retroviral gag protein is from Rous sarcoma virus (RSV).

In certain embodiments, the envelope protein comprises at least one heterologous polypeptide or fragment thereof. The envelope protein can further comprise transmembrane domains selected from those including any of haemagglutinin, Rous sarcoma virus (RSV) fusion protein, an E protein of tick borne encephalitis virus and dengue fever virus, the El protein of SFV, baculovirus gp64, and Vesicular stomatitis (Indiana) virus-G (VSV-G) protein, preferably a glycoprotein, or fragment or derivative thereof, more preferably from a RNA virus or a retrovirus, or fragment or derivative thereof, most preferably VSV-G or EnvA, or an alteration of VSV-G. In some embodiments the VLP described herein is not cytopathic to the target cell.

In certain embodiments, the target immune cell is a dendritic cell. In certain embodiments, the target immune cell can include any of the following cells: dendritic cells, macrophages, and any other antigen presenting cells.

In certain embodiments, the peptide VLP is capable of binding to a target cell of a subject, delivering the immunogenic antigen of the infectious agent and exposing the target cell to the antigenic determinants, which exposure is capable of inducing an immune response, including a T cell response, against coronavirus in the subject.

In certain embodiments, the immune response including a T cell response is induced by a dosing regimen comprising one or two administrations of the peptide VLP, wherein the dose is sufficient to induce an immune response against coronavirus in a subject.

In certain embodiments, the dosing regimen comprises one administration of the peptide VLP, wherein the one dose administration is sufficient to induce an immune response against the coronavirus in a subject. In certain embodiments, the immune response is effective to prevent or lessen COVID-19 infection for at least about 4 months, 5 months, 6 months, 7 months, 8 months, 9 months, 10 months, 11 months, 12 months, 13 months, 14 months, 15 months, 16 months, 17 months, or 18 months following a first vaccination with the peptide VLP vaccine. In certain embodiments, the dosing regimen includes at least one booster administration at about 4 months, 5 months, 6 months, 7 months, 8 months, 9 months, 10 months, 11 months, 12 months, 13 months, 14 months, 15 months, 16 months, 17 months, or 18 months following a first vaccination with the peptide VLP vaccine.

The VLPs described herein may be produced in a variety of ways, as will be apparent to those skilled in the art based on the provided disclosure. The commonality to these various methods is the expression of the described plasmids in a cell capable of expressing the necessary proteins (gag and a fusion protein). Accordingly, a method of producing a VLP described herein may include co-transforming, transfecting, or nucleofecting a eukaryotic cell with a plasmid comprising a polynucleotide sequence encoding at least one immunogenic antigen from an infectious agent; a plasmid comprising a polynucleotide sequence encoding a retroviral gag protein; and a plasmid comprising a polynucleotide sequence encoding a heterologous envelope protein; and culturing the co-transformed cell under conditions suitable to cause each plasmid to produce its encoded product, thereby producing a virus-like particle. In certain embodiments, one plasmid encodes the envelope protein which comprises least one antigen from an infectious agent and optionally one or more linkers or transmembrane domains and a cytoplasmic tail. In some embodiments the polynucleotide sequence encoding the gag protein is derived from Rous sarcoma virus. In some embodiments the polynucleotide sequence encoding the heterologous envelope protein encodes VSV-G transmembrane domain or a fragment thereof. The glycoprotein of the vesicular stomatitis virus (VSV-G) is a transmembrane protein that functions as the surface coat of the wild type viral particles and is an option to link the antigenic heterologous polypeptide or fragment thereof to the RSV capsid to form the peptide VLP particle.

### 4.4. Infectious Agents Encoded in the Peptide VLP

In certain embodiments, the infectious agent is a virus. In certain embodiments, the infectious agent is a bacterium. Exemplary bacteria include one or more of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

In additional embodiments, the virus is a coronavirus. In certain embodiments, the coronavirus is SARS-COV-2. In certain embodiments, the immunogenic antigen is a surface antigen. In additional embodiments, the peptide VLP is a SARS-COV-2 peptide VLP vaccine. In certain embodiments, the surface antigen has at least 80% identity to a coronavirus spike protein, coronavirus membrane protein, coronavirus hemagglutinin esterase (HE), coronavirus envelope protein, or any combination thereof, such as SEQ ID NO:4 (the ectodomain of SARS-CoV-2 spike glycoprotein fused with the HE domain). In certain embodiments, the surface antigen has at least 80% identity to any of these coronavirus spike proteins or combinations of epitopes or domains thereof.

In certain embodiments, the surface antigen has at least 80%, 85%, 90% or 95% identity to a coronavirus spike protein, coronavirus membrane protein, coronavirus hemagglutinin esterase (HE), coronavirus envelope protein, or any combination thereof, such as SEQ ID NO:4 (the ectodomain of SARS-COV-2 spike glycoprotein fused with the HA domain). In certain embodiments, the surface antigen has 80% identity to a coronavirus spike protein (SEQ ID NO:2) or has at least 80%, 85%, 90% or 95% identity to SEQ ID NO:4, or SEQ ID NOs:4-11. In certain embodiments, the spike protein sequence is encoded by a codon optimized sequence of SEQ ID NO: 1; or a codon optimized sequence of SEQ ID NO:3 encoding SEQ ID NO:4-11.

In certain embodiments, the SARS-COV-2 peptide VLP vaccine provided herein can prevent or decrease symptoms of the disease COVID-19. Such symptoms which may be lessened or prevented include any one or more of the following: fever, high temperature (>37.3° C.), cough, myalgia, sputum production, headache, hemoptysis, diarrhea, dyspnea and in some cases, acute respiratory distress syndrome (ARDS), acute cardiac injury or secondary infection. ARDS, caused by SARS-COV-2 is often associated with hypoxemia despite relatively normal lung function. The ALI is associated with cellular infiltration of the airways and inflammation. High mobility group box protein 1 (HMGB1) and interleukin-6 (IL-6) are among the pro-inflammatory cytokines implicated in ALI. Additional symptoms which may be prevented include those associated with acute respiratory distress syndrome (ARDS) and/or acute lung injury (ALI) and/or pneumonia.

### 4.5. Targeting Cells with a Peptide VLP Vaccine to Deliver SARS-COV-2 Antigens

Coronaviridae is a family of viruses (e.g., MERS-COV and Severe Acute Respiratory Syndrome (SARS-COV)) that primarily infect the upper respiratory and gastrointestinal tracts of mammals and birds, and that are responsible for acute and chronic diseases of the respiratory, hepatic, gastrointestinal and neurological systems. Coronaviruses are enveloped positive-sense, single-stranded RNA viruses with a nucleocapsid of helical symmetry and virions with a crown-like appearance. They have the largest genome among all RNA viruses, typically ranging from 27 to 32 kb. The genome is packed inside a helical capsid formed by the nucleocapsid protein (N) and further surrounded by an envelope. Associated with the viral envelope are at least three structural proteins: The membrane protein (M) and the envelope protein (E) are involved in virus assembly, whereas the spike protein (S) mediates virus entry into host cells. Some coronaviruses also encode an envelope-associated hemagglutinin-esterase protein (HE). Among these structural proteins, the spike forms large protrusions from the virus surface, giving coronaviruses the appearance of having crowns (hence their name; corona in Latin means crown). In addition to mediating virus entry, the spike is an important determinant of viral host range and tissue tropism and a major inducer of host immune responses.

The virions of each coronavirus are approximately 100 nm with a crown-like appearance because of the club-shaped spike (S) proteins projecting from the surface of the envelope. The spike protein is the viral membrane protein that is responsible for cell entry and includes an S1 domain, which is responsible for binding the cell surface receptor, and an S2 domain, which is a membrane-anchored subunit.

Upon entering an infected cell, coronaviruses transcribe their RNA and the viruses replicate in the cytoplasm of the infected cell. Replication is mediated by the synthesis of an antisense RNA strand, which is provided as a template for additional viral genomes and transcription. The viruses then assemble and released from the infected cell.

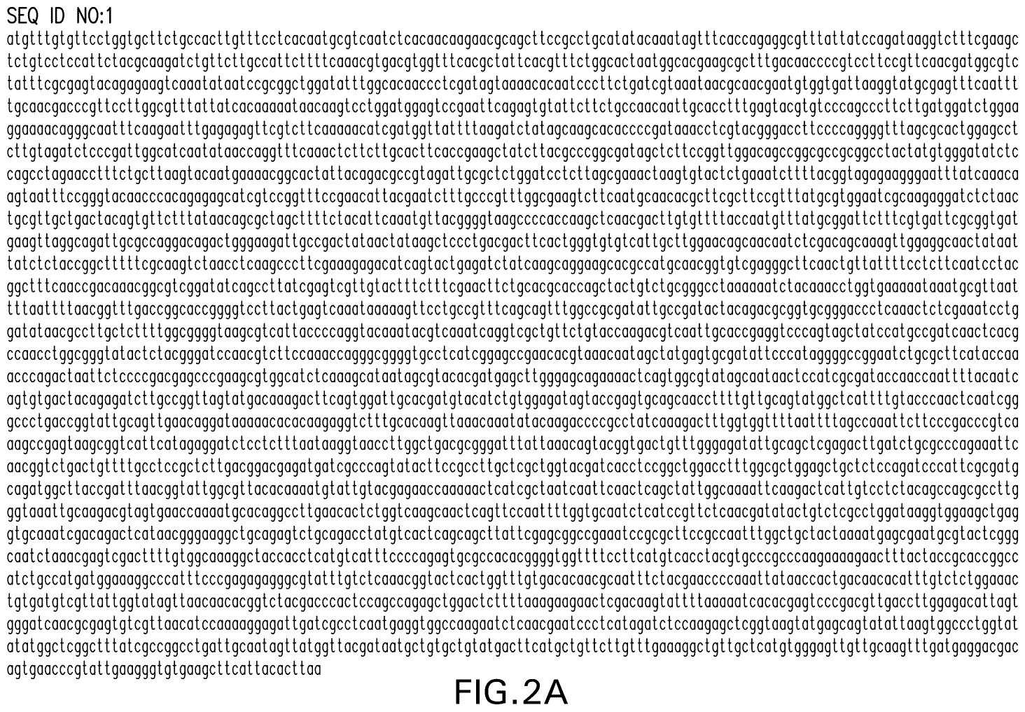

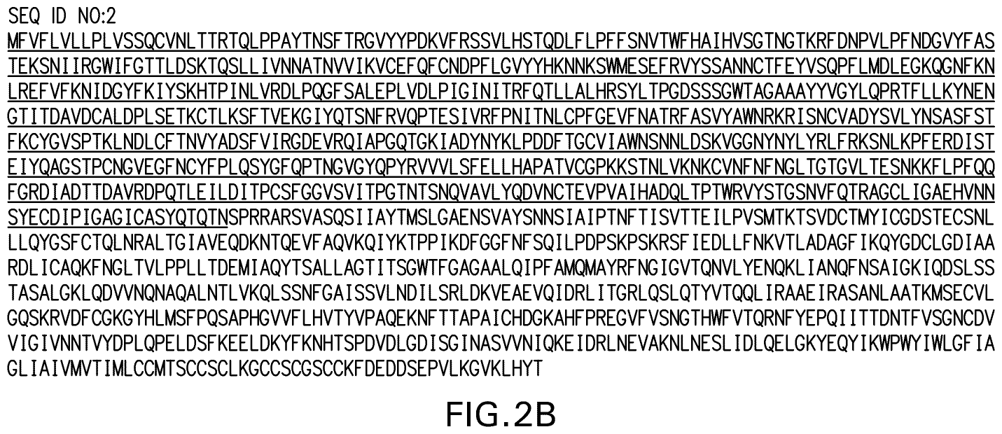

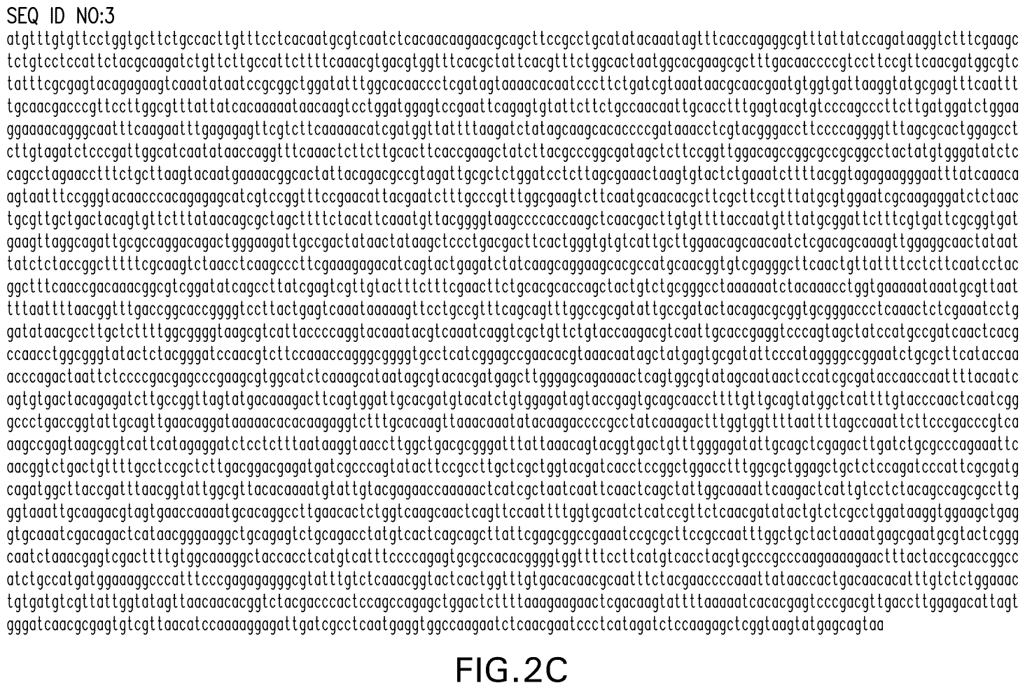

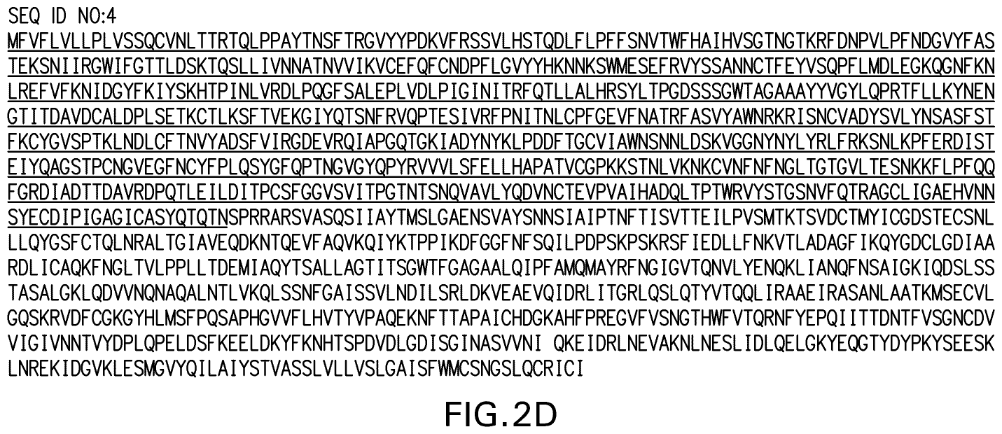

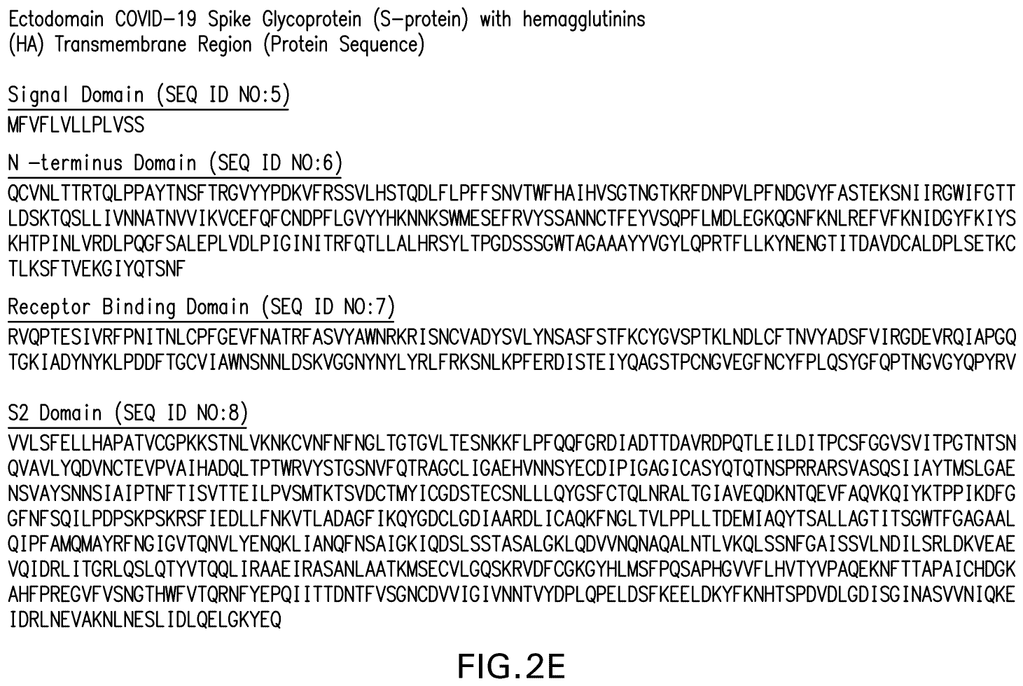

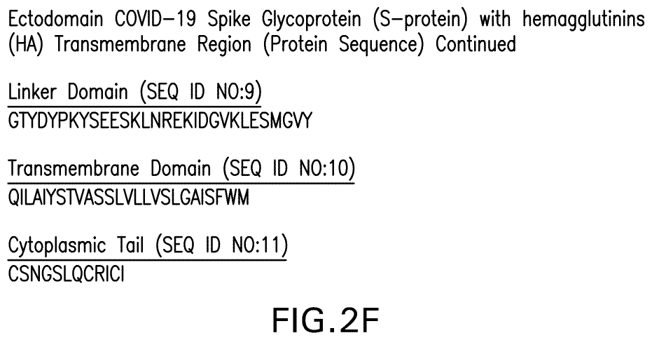

The amino acid sequence of the full length COVID-19 spike glycoprotein (S-protein) can be found at: Genbank ID 43740568. Underlined is the S1 domain in FIG. 2B.

SEQ ID NO: 2
MFVFLVLLPLVSSQCVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDS
KTQSLLIVNNATNVVIKVCEFQFCNDPFLGVYYHKNNKSWMESEFRVYSSANNCTF
EYVSQPFLMDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTFLLKYNENG
TITDAVDCALDPLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLF
RKSNLKPFERDISTEIYQAGSTPCNGVEGENCYFPLQSYGFQPTNGVGYQPYRVVVLS
FELLHAPATVCGPKKSTNLVKNKCVNFNFNGLTGTGVLTESNKKFLPFQQFGRDIAD
TTDAVRDPQTLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDFGGFNFSQI
LPDPSKPSKRSFIEDLLFNKVTLADAGFIKQYGDCLGDIAARDLICAQKFNGLTVLPPL
LTDEMIAQYTSALLAGTITSGWTFGAGAALQIPFAMQMAYRENGIGVTQNVLYENQ
KLIANQFNSAIGKIQDSLSSTASALGKLQDVVNQNAQALNTLVKQLSSNFGAISSVLN
DILSRLDKVEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDEDDSEPVL
KGVKLHYT

FIG. 2C shows the polynucleotide sequence (codon optimized) of the ectodomain COVID-19 spike glycoprotein (S-protein) with hemagglutinin (HA) transmembrane region from Influenza (SEQ ID NO:3). FIGS. 2D-2F show the amino acid sequence of the ectodomain COVID-19 spike glycoprotein (S-protein) fused with the hemagglutinin (HA) transmembrane region from influenza, which serves as an anchor in the VLP gag shell, along with the other amino acid domain sequences (SEQ ID NO:4-11).

### 4.6. Pharmaceutical Compositions

Additional aspects provided herein include a pharmaceutical composition comprising any of the peptide VLP s described herein.

In certain embodiments, the composition further comprises a pharmaceutically acceptable carrier, diluent, adjuvant and/or additive, or any combination thereof.

In certain embodiments, the pharmaceutical composition is capable of inducing an immune response against coronavirus in a mammalian subject.

In certain embodiments, wherein following administration of the composition to the subject, the peptide VLP is capable of inducing a T cell response against coronavirus in a mammalian subject.

In certain embodiments, the coronavirus is SARS-COV-2 and the VLP is s SARS-CoV-2 peptide VLP.

In certain embodiments, the VLP or pharmaceutical composition described herein is for use in diminishing or preventing at least one symptom of a coronavirus infection in a mammalian subject.

In certain embodiments, the diminishing or preventing comprises inducing coronavirus-specific immunity against SARS-COV-2 (COVID-19).

In certain embodiments, the peptide VLP or pharmaceutical composition described herein are for use in inducing cellular and or humoral immunity in a mammalian subject.

In certain embodiments, the peptide VLP or pharmaceutical composition described herein is for use in eliciting an immune response in a mammalian subject.

In certain embodiments, said inducing or eliciting an immune response is an immune response against SARS-COV-2 (COVID-19).

In certain embodiments, the peptide VLP or pharmaceutical compositions described herein are for use in inducing neutralizing antibodies against SARS-COV-2 in a mammalian subject.

### 4.7. Method of Diminishing or Preventing A Coronavirus Infection

Additional aspects provided herein include a method of diminishing or preventing a viral or bacterial infection in a mammalian subject comprising administering any of the peptide VLP's, or any of the pharmaceutical compositions described herein to the subject.

Additional aspects provided herein include a method of diminishing or preventing a coronavirus infection in a mammalian subject comprising administering any of the peptide VLP's, or any of the pharmaceutical compositions described herein to the subject.

Additional aspects provided herein include a method of inducing cellular and or humoral immunity against a coronavirus in a mammalian subject, comprising administering any of the peptide VLP's, or any of the pharmaceutical compositions described herein to the subject.

Additional aspects provided herein include a method of eliciting an immune response against a coronavirus in a mammalian subject, comprising administering any of the peptide VLP's, or any of the pharmaceutical compositions described herein to the subject.

In some embodiments, methods for increasing an immune response, or eliciting a desired immune response in a patient in need thereof are provided. In some embodiments, the methods comprise administering an effective amount of a peptide VLP's described herein, to the patient.

Diseases that the compositions and methods described herein can diminish or prevent microbial infections such as a viral infection, yeast infection, fungal infection, protozoan infection and/or bacterial infection.

Exemplary bacterial infections include those caused by one or more of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

By a “viral infection” is meant an infection caused by the presence of a virus in the body. Viral infections include chronic or persistent viral infections, which are viral infections that are able to infect a host and reproduce within the cells of a host over a prolonged period of time-usually weeks, months or years, before proving fatal. Viruses giving rise to chronic infections that which may be treated in accordance with the present invention include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include Epstein Barr virus (EBV), as well as other viruses such as those which may be associated with tumors.

Examples of viral infections which can be prevented or whose symptoms can be diminished by vaccination with the peptide VLP compositions and methods described herein include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus). The treatment and/or prevention of a viral infection includes, but is not limited to, preventing or alleviating one or more symptoms associated with said infection, the inhibition, reduction or suppression of viral replication and/or viral load, and/or the enhancement of the immune response.

In some embodiments, methods of inhibiting a viral or bacterial replication or reproduction in a subject having a viral or bacterial infection are provided. In some embodiments, the methods comprise administering to the subject with the viral or bacterial infection an effective amount of peptide VLP compositions to the patient. In some embodiments the viral or bacterial load in the patient is reduced or is undetectable. The viral or bacterial load can be reduced to undetectable levels or by at least 10, 20, 30, 40, 50, 60, 70, 80, 90, 91, 92, 93, 94, 95, 96, 96, 97, 98, or 99% as compared to the pre-treatment levels.

Additional aspects provided herein include a method of diminishing or preventing a coronavirus infection in a mammalian subject comprising administering the peptide VLP described herein to the subject.

Additional aspects provided herein include a method of inducing cellular and or humoral immunity in a mammalian subject, comprising administering any of the peptide VLP's described herein to the subject.

Additional aspects provided herein include a method of eliciting an immune response in a subject, comprising administering any of the peptide VLP's described herein to the subject.

Additional aspects provided herein include a method of inducing neutralizing antibodies against a coronavirus in a subject, comprising administering any of the peptide VLP's described herein to the subject.

In certain embodiments, the method further includes inducing a T cell response against the coronavirus. In certain embodiments, the T cell response is induced by a regimen comprising one or at least two administrations. In certain embodiments, the coronavirus is SARS-COV-2 (COVID-19).

Additional aspects provided herein include administering to a subject any of the peptide VLP's described herein, wherein the peptide VLP is capable of presenting the at least one heterologous polypeptide or fragment thereof to an antigen presenting cell in the subject, inducing a cellular and/or humoral immune response to SARS-COV-2 in the subject.

Additional aspects provided herein include a method of inducing neutralizing antibodies against SARS-COV-2 in a mammalian subject, comprising administering any of the peptide VLP's, or any of the pharmaceutical compositions described herein to the subject.

In certain embodiments, the method further includes inducing a T cell response against the coronavirus.

In certain embodiments, the immune response is induced by a regimen comprising one or two administrations of any of the peptide VLP or pharmaceutical compositions described herein.

In certain embodiments, the immune response is induced by a regimen comprising one administration of any of the peptide VLP s or pharmaceutical compositions described herein. In certain embodiments, the coronavirus is SARS-COV-2.

Additional aspects provided herein include a method of expressing a recombinant polynucleotide encoding an immunogenic antigen of an infectious agent in a subject, comprising administering any of the peptide VLP s described herein, or any of the pharmaceutical compositions described herein to the subject.

In certain embodiments, the peptide VLP is capable of binding to a target cell, after which the target cell is capable of expressing the immunogenic antigen of the infectious agent, which expression is capable of inducing a cellular and/or humoral immune response to SARS-CoV-2 in the subject.

### 4.8. Peptide VLP Composition Stability

Additional aspects provided herein include wherein any of the peptide VLP or compositions thereof is stable at from the range of about 4° C.-10° C. for at least about one-six months. In certain embodiments, the peptide VLP is stable for at least about six to nine months. In certain embodiments, the peptide VLP is stable for at least about nine to twelve months. Additional embodiments include wherein the peptide VLP is stable for 2° C.-8° C. for at least one-six months. Additionally, in certain embodiments, the peptide VLP is stable at about −80 C for at least 2 years.

### 4.9. Production of Peptide VLPs

Additional aspects provided herein include a method of producing any of the SA-RNA VLPs described herein, comprising:
co-transforming a eukaryotic cell with:
   a first vector comprising a polynucleotide sequence encoding a retroviral gag protein (the capsid protein); and
   a second vector comprising a polynucleotide sequence encoding the envelope protein which comprises a heterologous polypeptide or fragment thereof (which binds to the capsid in a self-assembling manner);
b. culturing the co-transformed eukaryotic cell under conditions suitable to cause each vector to produce its encoded product, thereby producing the peptide VLP; and
c. isolating the peptide VLP from the eukaryotic cell.

Additional aspects provided herein include a peptide VLP produced by any of the methods described herein.

A plasmid useful in the production of the VLPs described herein is a plasmid that encodes a virus structural protein. One such class of proteins is the retroviral group-specific antigen (gag) protein. The gag protein is the core structural protein of retroviruses and, in some instances, is capable of forming enveloped virus cores when expressed in eukaryotic cells. This property makes gag proteins particularly useful in the production of VLPs, because they can form the basic structural aspect of the particle and allow for packaging of RNA associated with a retroviral packaging signal sequence. Accordingly, described herein are plasmids that include a polynucleotide that encodes a retroviral gag protein. In some embodiments, the described plasmids include a polynucleotide that encodes a retroviral gag protein and a promoter polynucleotide sequence that allows for the gag gene sequence to be transcribed into mRNA by host cell RNA polymerase. In one embodiment, the promoter polynucleotide sequence is derived from a virus, such as SV40 or CMV. In some embodiments, the plasmid will further include a polynucleotide that encodes a heterologous polypeptide or fragment of interest. Those skilled in the relevant art will understand that a polynucleotide sequence of a gag protein from any retrovirus may be used to produce the plasmids and VLPs described herein. In some embodiments the polynucleotide sequence encoding the gag protein may be derived from Rous sarcoma virus. In some embodiments the polynucleotide sequence encoding the gag protein may be derived from murine leukemia virus. In some embodiments the polynucleotide sequence encoding the gag protein may be derived from SIV. In some embodiments the polynucleotide sequence encoding the gag protein may be derived from human T-lymphotropic virus.

Another plasmid useful in the production of the VLPs described herein is a plasmid that encodes a protein to mediate fusion between the VLP envelope and a production host cell. A class of proteins suitable for this purpose is viral fusion proteins, which facilitate virus infection of cells by allowing an enveloped virus to fuse its membrane with that of a host cell. Many of viral fusion proteins also have known, or suspected, cellular receptor proteins that may allow for targeting of selected cell types, or in cases of more ubiquitous receptors, such as sialic acid for influenza virus, more generalized targeting may be desired. In some instances, viral fusion proteins work in conjunction with viral attachment proteins, ligands for cellular receptor, a receptor for a cell ligand, or accessory proteins, thus proteins of this sort may also be encoded by the described plasmids, in addition to, or also by, the plasmid encoding a viral fusion protein. Alternatively, in some embodiments a viral fusion protein from one virus may be encoded by the described plasmid along with a viral attachment protein of another virus, a ligand of a cellular receptor, a receptor of a cell ligand, or an accessory protein to facilitate, or direct, targeting of a VLP to a desired cell type. In some embodiments the viral fusion protein, viral attachment protein, ligand of a cellular receptor, receptor of a cell ligand, or accessory protein will be a type-I membrane protein, which will allow the extracellular domain of the protein to be oriented extracellularly when present on the cell surface. This will also allow the fusion protein to be correctly oriented following budding of a VLP from a packaging cell. Expression of such proteins in a cell will typically result in the cell surface being coated with the proteins, so that budding of a VLP from any part of the cell membrane will provide the VLP with some amount of the protein(s) on its surface. In some embodiments, the described plasmids include a polynucleotide that encodes a viral fusion protein and a promoter polynucleotide sequence that allows for the fusion protein gene sequence to be translated into mRNA by host cell RNA polymerase. In one embodiment, the promoter polynucleotide sequence is derived from a virus, such as SV40 or CMV. In some embodiments, the described plasmids include a polynucleotide that encodes a viral attachment protein and a promoter polynucleotide sequence that allows for the attachment protein gene sequence to be translated into mRNA by host cell RNA polymerase. In one embodiment, the promoter polynucleotide sequence is derived from a virus, such as SV40 or CMV. In some embodiments the plasmids described herein include a polynucleotide that encodes a vesicular stomatitis virus G protein. In some embodiments the plasmids described herein include a polynucleotide that encodes the influenza hemagglutinin protein. In some embodiments the plasmids described herein include a polynucleotide that encodes the influenza neuraminidase protein. In some embodiments the plasmids described herein include a polynucleotide that encodes the respiratory syncytial virus fusion protein. In some embodiments the plasmids described herein include a polynucleotide that encodes the rotavirus VP7 protein. Other such fusion proteins will be apparent to those skilled in the art based on desired tropism or cell target of the associated virus.

Provided herein are cells comprising the plasmids described to produce VLPs. These cells may be used to produce the VLPs described herein by transcribing or expressing the polynucleotides of the plasmids. For instance, a mammalian cell transfected with a plasmid encoding a retroviral gag protein, and a plasmid encoding an antigenic protein could produce a peptide VLP having the desired antigenic protein on its surface, which will serve as a peptide VLP vaccine.

The described cells may be any eukaryotic cell capable of transcribing, and where necessary (such as in the case of the gag and fusion proteins), translating the polynucleotides of the described plasmids. The cells will likely be mammalian cells in many embodiments. For example, rodent cells, such as murine, hamster (CHO or BHK-21), or rat cells could be used to express the described plasmids; canine cells, such as Madin Darby canine kidney cells, could be used to express the described plasmids; primate cells, such as vero cells, could be used to express the described plasmids; and human cells, such as HEK293T cells (human kidney), Hep-2 cells (human airway), Caco-2 (intestine), HeLa (epithelium), and other such cell lines known in the art, could be used to express the described plasmids. In some embodiments the described cells can be transfected and selected, using standard transfection and selection methods known in the art, to stably comprise one or more of the described plasmids.

In some embodiments the production cell lines described herein will contain a plasmid comprising a polynucleotide sequence encoding a gag protein, and a plasmid comprising a polynucleotide sequence encoding a heterologous envelope protein, wherein neither the plasmids nor the cell contain a gene encoding an alphavirus structural protein. In some embodiments the polynucleotide sequence encoding the gag protein is derived from Rous sarcoma virus. In some embodiments the polynucleotide sequence encoding the heterologous envelope protein encodes a transmembrane VSV-G or other transmembrane elements or linkers. In certain embodiments, the envelope protein comprises a heterologous polypeptide or fragment thereof, which may be an immunogenic surface antigen, such as the ectodomain of SARS-COV-2 spike glycoprotein fused with the HA domain. In certain embodiments, the surface antigen has 80% identity to a coronavirus spike protein (SEQ ID NO:2) or to the ectodomain of SARS-COV-2 spike glycoprotein fused with the HA domain (SEQ ID NO:4-11), which serves as an anchor to the gag shell (See, FIG. 1A).

### 4.10. Pharmaceutical Compositions

Described herein are compositions comprising at least one described VLP and a pharmaceutically acceptable carrier. Such compositions are useful, for example, for administration to subjects in need of expression of an exogenous protein or increased expression of a protein normally found in those of the same species as the subject. The compositions may be formulated as any of various preparations that are known and suitable in the art, including those described and exemplified herein. In some embodiments, the compositions are aqueous formulations. Aqueous solutions may be prepared by admixing the VLPs in water or suitable physiologic buffer, and optionally adding suitable colorants, flavors, preservatives, stabilizing and thickening agents and the like as desired. Aqueous suspensions may also be made by dispersing the VLPs in water or physiologic buffer with viscous material, such as natural or synthetic gums, resins, methylcellulose, sodium carboxymethylcellulose, and other well-known suspending agents.

The compositions may be formulated for injection into a subject. For injection, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol, before use.

The compositions may be formulated for aerosolized delivery to a subject. For aerosol delivery, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents.

The compositions may be formulated in sustained release vehicles or depot preparations. Such long-acting formulations may be administered by implantation (for example subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples of delivery vehicles suitable for use as carriers for hydrophobic drugs.

Coronaviruses are large, enveloped, positive-stranded RNA viruses. They have the largest genome among all RNA viruses, typically ranging from 27 to 32 kb. The genome is packed inside a helical capsid formed by the nucleocapsid protein (N) and further surrounded by an envelope. Associated with the viral envelope are at least three structural proteins: The membrane protein (M) and the envelope protein (E) are involved in virus assembly, whereas the spike protein (S) mediates virus entry into host cells. Some coronaviruses also encode an envelope-associated hemagglutinin-esterase protein (HE). Among these structural proteins, the spike forms large protrusions from the virus surface, giving coronaviruses the appearance of having crowns (hence their name; corona in Latin means crown) (FIGS. 1B and 2A). In addition to mediating virus entry, the spike is an important determinant of viral host range and tissue tropism and a major inducer of host immune responses.

In certain aspects, provided herein is a recombinant alphavirus VLP particles, expressing on its surface at least one SARS-COV-2 S glycoprotein (NCBI Reference Sequence: NC 045512.2; Protein_ID: YP_009724390.1; SEQ ID NO: 2) or the ectodomain of SARS-CoV-2 spike glycoprotein fused with the HA domain (SEQ ID NO:4-11), or a fragment or derivative thereof. See FIGS. 1A-1B and FIGS. 2A-2F; The fragment may be derived from any of the known regions of SARS-COV-2 S glycoprotein, such as S1, S2, or the ectodomain, or the RBD, or combinations thereof (see Walls et al., Cell, published online Mar. 9, 2020; available at doi.org/10.1016/j.cell.2020.02.058).

Additionally, any immunogenic antigen could be similarly anchored into the VLP gag shell on the envelope protein, using a hemagglutinin anchor alone or in combination with a linker and optionally including a cytoplasmic tail from the hemagglutinin, as described herein. Such immunogenic antigens can include surface proteins, lipopolysaccharides, and peptidoglycans on the bacterial cell wall. An example of such an antigen includes pili, lipopolysaccharides, various cell wall components, and flagella (See, Immunology for Pharmacy, 2021 pgs 147-151 https://doi.org/10.1016/B978-0-323-06947-2.10018-5).

### 4.11. Coronavirus Variants

In some embodiments, the virus is a coronavirus. In certain embodiments, the virus is any one or combination of the following coronaviruses: coronavirus OC43, coronavirus 229E, coronavirus NL63, coronavirus HKU1, middle east respiratory syndrome beta coronavirus (MERS-COV), severe acute respiratory syndrome beta coronavirus (SARS-COV), and SARS-CoV-2 (COVID-19). In a specific embodiment, the virus is SARS-COV-2. In particular embodiments, the patient has severe acute respiratory syndrome (SARS). In particular embodiments, the patient has middle eastern respiratory syndrome (MERS). In particular embodiments, the patient has coronavirus disease 2019 (COVID-19). In particular embodiments, the VLP is capable of eliciting an immune reaction that is protective against coronavirus variants.

The percentage amino acid identity of the coronavirus spike and nucleocapsid proteins to SARS-COV-2 Proteins are shown in Table1 (See, Okba et al. Emerging Infectious Diseases (2020) 26:7 1478-1488).

TABLE 1

| | | | | S1 (N- | S2 (C- |
|---|---|---|---|---|---|
| Virus Type | Virus | Nucleocapsid | S | terminal) | terminal) |
| Betacoronavirus | SARS-CoV | 90 | 77 | 66 | 90 |
| | MERS- | 49 | 33 | 24 | 43 |
| | CoV | | | | |
| | HCoV- | 34 | 33 | 25 | 42 |
| | OC43 | | | | |
| | HCoV- | 34 | 32 | 25 | 40 |
| | HKU1 | | | | |
| Alphacoronavirus | HCoV- | 28 | 30 | 24 | 35 |
| | 229E | | | | |
| | HCoV- | 29 | 28 | 21 | 36 |
| | NJ63 | | | | |

The VLP can be pseudotyped to express different surface epitopes to elicit an immune response. The coronavirus has immunogenic antigens/polypeptides including at least: envelope proteins, membrane proteins, spike proteins, and hemagglutinin. The immunogenic antigen can be a sequence that is at least 70% homologous or exhibits at least 70% identity to any of these polypeptides, fragments thereof, or epitopes from a coronavirus. Most preferably, the virus is SARS-COV-2.

Multiple variants of SARS-COV-2 have arisen throughout the world in the eighteen months since the virus was first detected. Three mutations in particular, B.1.1.7, B.1.351, and P.1, have become dominant and have increased the effectiveness of the virus. The N501Y mutation, which is in the receptor-binding-domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity).

The peptide COVID VLP vaccines described herein can be adapted to encode any variant of the SARS-COV-2 spike protein (based on reference protein found at Genbank: NC_045512), or any antigenic protein desired to be utilized as an “immune targeting” agent to stimulate host anti SARS-COV-2 antibody production. Additionally, a surprising observation is that an exemplary peptide VLP, CB-109 is capable of inducing a cellular and/or humoral immune response to Severe Acute Respiratory Syndrome (SARS-COV), and SARS-COV-2, including inducing an immune response against the South African, UK and Brazilian variants of SARS-COV-2) in test subjects. Thus, illustrating the flexibility and widely applicable platform of utilizing the peptide VLP's as described herein for generating protective immune responses.

A listing of emerging variants can be found online at: covariants.org. and are summarized in the table below.

TABLE 2

| Variant | First detected | Key mutations |
|---|---|---|
| B.1.526 | New York/November 2020 | S: L5F |
| 20C/S: 484K | | S: T95I |
| | | S: D253G |
| | | S: E484K |
| | | S: A701V |
| | | ORF1b: Q1011H |
| | | ORF3a: P42L |
| | | ORF1a: S3675— |
| | | ORF1a: G3676— |
| | | ORF1a: F3677— |
| | | N: P199L |
| | | N: M234I |
| | | ORF8: T11I |
| | | C241T |
| | | C3037T |
| | | A20262G |
| | | A28271— |
| B.1.525 | New York/December 2020 | S: A67V |
| 20A/S: 484K | | S: H69— |
| | | S: V70— |
| | | S: Y144— |
| | | S: E484K |
| | | S: Q677H |
| | | S: F888L |
| | | N: S2— |
| | | N: D3Y |
| | | N: A12G |
| | | ORF1a: S3675— |
| | | ORFla: G3676— |
| | | ORF1a: F3677— |
| | | C241T |
| | | C1498T |
| | | A1807G |
| | | G2659A |
| | | C3037T |
| | | T8593C |
| | | C9593T |
| | | C18171T |
| | | A20724G |
| | | C24748T |
| | | A28699G |
| | | G29543T |
| P.2 | Brazil/April 2020 | Spike: E484K, |
| | | D614G, V1176F |
| | | ORF1a: L3468V, |
| | | L3930F |
| | | ORF1b: P314L |
| | | N: A119S, R203K, |
| | | G204R, M234I |
| | | 5′UTR: R81C |
| B.1.1.7 | United Kingdom | Δ69/70 |
| 201/501Y.V1 | | Δ144Y |
| | | (E484K*) |
| | | (S494P*) |
| | | N501Y |
| | | A570D |
| | | D614G |
| | | P681H |
| | | C241T |
| | | C913T |
| | | C3037T |
| | | C5986T |
| | | C14676T |
| | | C15279T |
| | | T16176C |
| | | ORF1a: S3675— |
| | | ORF1a: G3676— |
| | | ORF1a: F3677— |
| | | N: R203K |
| | | N: G204R |
| | | ORF8: Q27* |
| P.1 | Japan/ | S: L18F |
| 20J/501Y.V3 | Brazil | S: K417T |
| | | S: E484K |
| | | S: N501Y |
| | | S: H655Y |
| | | ORF1a: S3675— |
| | | ORF1a: G3676— |
| | | ORF1a: F3677— |
| | | N: P80R |
| | | N: R203K |
| | | N: G204R |
| | | D614G |
| | | C241T |
| | | T733C |
| | | C2749T |
| | | C3037T |
| | | A6319G |
| | | A6613G |
| | | C12778T |
| | | C13860T |
| | | A28877T |
| | | G28878C |
| B.1.351 | South Africa | D80A |
| 20H/501Y.V2 | | S: D215G |
| | | S: L241— |
| | | S: L242— |
| | | S: A243— |
| | | S: K417N |
| | | S: E484K |
| | | S: N501Y |
| | | S: A701V |
| | | ORF1a: S3675— |
| | | ORF1a: G3676— |
| | | ORF1a: F3677— |
| | | N: T205I |
| | | E: P71L |
| | | D614G |
| | | G174T |
| | | C241T |
| | | C3037T |
| | | C28253T |
| B.1.427 | US-California | L452R |
| | | D614G |
| B.1.429 | US-California | S: S13I |
| 20C/S: 452R | | S: W152C |
| | | S: L452R |
| | | ORF1a: I4205V |
| | | ORF1b: D1183Y |
| | | D614G |
| | | C2395T |
| | | T2597C |
| | | T24349C |
| | | G27890T |
| | | A28272T |
| 20E (EU1) | Spain-EU | S: A222V |
| | | ORF10: V30L |
| | | N: A220V |
| | | T445C |
| | | C6286T |
| | | C26801G |
| 20A EU2 | EU | S: S477N |
| | | N: M234I |
| | | N: A376T |
| | | ORF1b: A176S |
| | | ORF1b: V767L |
| | | ORF1b: K1141R |
| | | ORF1b: E1184D |
| | | C4543T |
| | | G5629T |
| | | C11497T |
| | | T26876C |
| 20A/S: 439K | Ireland/Scotland | S: S439K |
| | | ORF1a: I2501T |
| | | C8047T |
| S: 677H.Robin1 | Wisconsin- US | S: Q677H |
| | | N: P67S |
| | | N: P199L |
| | | N: D377Y |
| | | ORF1a: T265I |
| | | ORF1a: L3352F |
| | | ORF1b: N1653D |
| | | ORF1b: R2613C |
| | | C241T |
| | | G1942T |
| | | C3037T |
| | | A9085G |
| | | C14805T |
| S: 677P.Pelican | US | S: Q677P |
| | | N: P67S |
| | | N: P199L |
| | | ORF1a: T265I |
| | | ORF1a: L3352F |
| | | ORF1b: N1653D |
| | | ORF1b: R2613C |
| | | C241T |
| | | C3037T |
| | | C21811A |
| | | T29194C |
| | | T29377A |
| 20A/S: 98F | Belgium and the Netherlands | S: S98F |
| | | N: P199L |
| | | ORF3a: Q38R |
| | | ORF3a: G172R |
| | | ORF3a: V202L |
| | | C28651T |
| 20C/S: 80Y | France/EU | S: D80Y |
| | | N: S186Y |
| | | N: D377Y |
| | | ORF1a: T945I |
| | | ORF1a: T1567I |
| | | ORF1a: Q3346K |
| | | ORF1a: V3475F |
| | | ORF1a: M3862I |
| | | ORF1b: P255T |
| | | ORF7a: R80I |
| | | G4960T |
| | | C6070T |
| | | C7303T |
| | | C7564T |
| | | C10279T |
| | | C10525T |
| | | C10582T |
| | | C27804T |
| 20B/S: 626S | Norway | S: 626S |
| 20B/S: 1122L | Sweden | S: V1122L |

### 4.12. Immunogenic and/or Antigenic Compositions and Vaccines and Administration

In one aspect, the disclosure provides a recombinant VLP particle that is capable of directing expression of a heterologous or foreign protein (e.g., a coronavirus protein) in the host or patient/subject as VLP vaccine.

In certain embodiments, an immunogenic and/or antigenic composition or vaccine is formulated such that the VLP vaccine, in which the RNA in the genome directs the production of at least one immunogenic antigen of an infectious agent in a target/host cell so as to elicit an immune (humoral and/or cell mediated) response in the target/host that is prophylactic or therapeutic. In an embodiment wherein the peptide VLP delivers an RNA encoding at least one immunogenic antigen of a pathogen (e.g., SARS-Cov-2), administration of the peptide VLP vaccine is carried out to prevent or treat an infection by the pathogen and/or the resultant infectious disorder and/or other undesirable correlates of infection.

In certain aspects, provided herein are compositions (e.g., pharmaceutical compositions, immunogenic compositions, VLP vaccines) comprising the recombinant VLP particles described herein and a carrier and/or excipient.

Administration of the recombinant VLP particles described herein can be used as a method of immunostimulation, to boost the host's immune system, enhancing cell-mediated and/or humoral immunity, and facilitating the clearance of infectious agents or symptoms of a disease or disorder in a subject infected with SARS-COV-2 (e.g., having COVID-19). The present disclosure thus provides a method of immunizing an animal or treating or preventing various diseases or disorders in an animal, comprising administering to the animal an effective immunizing dose of a vaccine of the present disclosure.

In certain aspects, the disclosure provides a method of treating or preventing a disease or disorder in a subject comprising administering to the subject an effective amount of the recombinant VLP particles described herein to induce an immune response (e.g., a protective immune response) against a foreign protein. In certain embodiments, the foreign protein is a coronavirus spike ectodomain, or a fragment or a derivative thereof. In a specific embodiment, the spike ectodomain is derived from SARS-COV-2.

In certain embodiments, the disclosure provides a method for the treatment or prevention of a disease or disorder in a subject infected with SARS-COV-2. In certain embodiments, the disease or disorder is COVID-19.

In certain aspects, the disclosure provides a method of treating or preventing a disease or disorder in a subject comprising administering to the subject an effective amount of the recombinant VLP particles described herein to induce the formation of neutralizing antibodies against a foreign protein. In certain embodiments, the foreign protein is a coronavirus S glycoprotein, or a fragment or a derivative thereof. In a specific embodiment, the S glycoprotein is derived from SARS-COV-2. In certain embodiments, the disclosure provides a method for the treatment or prevention of a disease or disorder in a subject infected with SARS-CoV-2. In certain embodiments, the disease or disorder is COVID-19.

In certain embodiments directed to vaccines, the recombinant VLP particles described herein are administered prophylactically, to prevent/protect against a SARS-COV-2 infection and/or infectious disease (e.g., having COVID-19).

Many methods may be used to introduce the immunogenic and/or antigenic compositions and vaccines described herein, such as, but not limited to oral, intradermal, intramuscular, intraperitoneal, intravenous, infusions, subcutaneous, intranasal routes, and via scarification (scratching through the top layers of skin, e.g., using a bifurcated needle).

In certain embodiments, the delivery route is intramuscular (IM). The muscles have a plentiful supply of blood, which helps ensure that the body absorbs the medication quickly. The tissue in the muscles can also hold more medication than fatty tissue. In certain embodiments, intramuscular injection is followed by electroporation.

The subject to which the VLP vaccine is administered can include humans, non-human primates, veterinary animals (e.g., cats, dogs, cows, horses, sheep, pigs, goats, hamsters, etc.), and experimental animal models of diseases (e.g., mice, rats, ferrets, monkeys, etc.). In a preferred embodiment, the subject is a human.

The VLP vaccines described herein comprise an effective amount of one or more recombinant VLP particles described herein) and a pharmaceutically acceptable carrier or excipient. Pharmaceutically acceptable carriers are well known in the art and include, but are not limited to, saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration, which is readily determined by one of skill in the art.

In certain embodiments, the VLP vaccine can also comprise minor amounts of wetting or emulsifying agents, or pH buffering agents. The immunogenic and/or antigenic composition or vaccine can be a liquid solution, suspension, emulsion, tablet, pill, capsule, sustained release formulation, or powder. Oral formulations can include one or more standard carriers such as pharmaceutical grades of mannitol, lactose, starch, gelatin, magnesium stearate, sodium saccharine, cellulose, magnesium carbonate, methylcellulose (e.g., 4000 cP, 25 cP, METHOCEL™ E3, E5, E6, E15, E50, E4M, E10M, F4, F5, F4M, K3, K100, K4M, K15M, K100M, K4M CR, K15M CR, K100M CR, E4M CR, E10M CR, K4M Premium, K15M Premium, K100M Premium, E4M Premium, E10M Premium, K4M Premium CR, K15M Premium CR, K100M Premium CR, E4M Premium CR, E10M Premium CR, and K100 Premium LV), monosodium glutamate, human serum albumin, fetal bovine serum, trehalose, alginate (e.g., BioReagent), guar gum, MUCOLOX™, etc. In certain embodiments, the formulation has an appropriate viscosity to maintain stability of the virus particles. In certain embodiments, the formulation has an appropriate carrier to allow the viral particles to maintain contact with mucosal membranes for an appropriate amount of time for them to be taken up.

The ingredients are supplied either separately or mixed together in unit dosage form, for example, as a dry lyophilized powder or water free concentrate in a hermetically sealed container such as an ampoule or sachette indicating the quantity of active agent. In certain embodiments where in the immunogenic and/or antigenic composition or vaccine is administered by injection, an ampoule of sterile diluent can be provided so that the ingredients may be mixed prior to administration.

In certain embodiments, lyophilized recombinant VLP particles described herein are provided in a first container and a second container comprises diluent (e.g., an aqueous solution of 50% glycerin, 0.25% phenol, and an antiseptic (e.g., 0.005% brilliant green)).

The precise dose of virus, or subunit vaccine, to be employed in the VLP vaccine will also depend on the route of administration, and the nature of the patient, and should be decided according to the judgment of the practitioner and each patient's circumstances according to standard clinical techniques. The VLP vaccine is administered in an amount sufficient to produce an immune response to the foreign protein in the host to which the recombinant VLP particle is administered.

In certain embodiments, the immunogenically and/or antigenically effective amount can comprise a dosage of about of 1 μg-1000 μg of total protein equivalent of the VLP which is effective for generating antibodies to the antigen. In certain embodiments, effective doses of the immunogenic and/or antigenic composition or vaccine described herein may also be extrapolated from dose-response curves derived from animal model test systems. Effective dosages include: 1 μg, 2 μg, 3 μg, 4 μg, 5 μg, 6 μg, 7, μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg; 110 μg; 120 μg; 130; 140 μg; 150; 160 μg; 170 μg; 180 μg; 190 μg; 200 μg; 210 μg; 220 μg; 230 μg; 240 μg; 250 μg; 260 μg; 270 μg; 280 μg; 290 μg; 300 μg; 310 μg; 320 μg; 330 μg; 340 μg; 350 μg; 360 μg; 370 μg; 380 μg; 390 μg; 400 μg; 410 μg; 420 μg; 430 μg; 440 μg; 450 μg; 460 μg; 470 μg; 480 μg; 490 μg; 500 μg; 510 μg; 520 μg; 530 μg; 540 μg; 550 μg; 560 μg; 570 μg; 580 μg; 590 μg; 600 μg; 610 μg; 620 μg; 630 μg; 640 μg; 650 μg; 660 μg; 670 μg; 680 μg; 690 μg; 700 μg; 710 μg; 720 μg; 730 μg; 740 μg; 750 μg; 760 μg; 770 μg; 780 μg; 790 μg; 800 μg; 810 μg; 820 μg; 830 μg; 840 μg; 850 μg; 860 μg; 870 μg; 880 μg; 890 μg; 900 μg; 910 μg; 920 μg; 930 μg; 940 μg; 950 μg; 960 μg; 970 μg; 980 μg; 990 μg; and 1000 μg. In some embodiments, a dose of at least about >20 μg is optimal. Such effective dose is considered a very low dose, and the ability of such a low dose, including a dose of >10 ng, to elicit a protective immune response in a patient/subject is unexpected.

In certain embodiments, a boosting dose is used. In certain embodiments, the boosting dose can be any SARS-COV-2 vaccine. In certain embodiments, the boosting dose comprises any of the recombinant VLP particle vaccines described herein. In certain embodiments, the boosting dose comprises the foreign protein or peptide in purified form, or a nucleic acid encoding the foreign protein or peptide, rather than using a recombinant VLP particle described herein. In certain embodiments, the boosting dose comprises the same SARS-COV-2 vaccine as the SARS-COV-2 vaccine it is boosting. In certain embodiments, the boosting dose comprises a SARS-COV-2 vaccine that is different than the SARS-COV-2 vaccine it is boosting.

In certain embodiments, the boosting dose comprises any of the recombinant VLP particle vaccines described herein. In certain embodiments, the boosting dose is used to boost any of the recombinant VLP particle vaccines described herein. In certain embodiments, the boosting dose is used to boost a SARS-COV-2 vaccine other than the recombinant VLP particle vaccines described herein. In certain embodiments, the vaccines can be heterologous.

Many methods may be used to introduce the boosting dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the delivery route is intramuscular.

In certain embodiments, the boosting dose is administered after a single dose of the SARS-COV-2 vaccine. In certain embodiments, boosting dose is administered after repeated doses of the SARS-COV-2 vaccine (e.g., 2, 3, 4, or 5 doses). The period of time between SARS-COV-2 vaccine administration and the boosting dose can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. If more than one boost is performed, the subsequent boost can be administered 1 week, 2 weeks, 3 weeks, 4 weeks, 5 weeks, 6 weeks, 7 weeks, 8 weeks, 3 months, 4 months, 5 months, 6 months, or longer after the preceding boost. For example, the interval between any two boosts can be 4 weeks, 8 weeks, or 12 weeks. For example, the SARS-COV-2 vaccine may be administered twice (e.g., via injection) before the boosting dose is administered (e.g., orally) and the boost is repeated every 3 months.

In certain embodiments, the priming dose comprises any of the recombinant VSV particle vaccines described herein. In certain embodiments, the priming dose is used to prime any of the recombinant VLP vaccines described herein. In certain embodiments, the priming dose is used to prime a SARS-COV-2 vaccine other than any of the recombinant peptide VLP vaccine described herein. In certain embodiments, the priming dose comprises the same SARS-COV-2 vaccine as the SARS-COV-2 vaccine it is priming. In certain embodiments, the priming dose comprises a SARS-COV-2 vaccine that is different than the SARS-COV-2 vaccine it is priming.

An advantage of the present peptide VLP system is that it is not limiting for additional treatments-be they AAV based, or other RNA based vaccines. The peptide VLP backbone does not produce substantial cross-reacting antibodies, that would inhibit or affect administration of vaccines that use a different platform or backbone. Additionally, there are no limitations on future vaccines or treatment using the peptide VLP system or platform. Thus, the present peptide VLP vaccines can be utilized with any heterologous boost vaccine as desired.

Many methods may be used to introduce the priming dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the priming dose is administered via intramuscular injection.

The period of time between the VLP priming dose and VLP vaccine administration can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. For example, the interval between the VLP priming dose and the vaccine can be 4 weeks, 8 weeks, or 12 weeks. For example, the VLP priming dose may be administered (e.g., via injection) before the VLP vaccine is administered. In additional embodiments, the interval between any two boosts can be 4 weeks, 8 weeks, or 12 weeks. For example, peptide VLP vaccine may be administered twice (e.g., via injection) before the boosting dose is administered (e.g., orally) and the boost is repeated every 3 months.

### 4.13. Stability of Peptide VLP Vaccine Compositions

In certain aspects, the disclosure provides VLP compositions that are stable at from about 4°-10° C. In certain embodiments, the VLP compositions are stable at from about 4°-10° C. to at least about one week, at least about ten days, at least about two weeks, at least about three weeks, at least about four weeks, at least about five weeks, at least about six weeks, at least about seven weeks, at least about eight weeks, at least about nine weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, at least about 12 months, or at least about 2 years.

In another aspect, the disclosure provides VLP compositions that allow at least 3 freeze/thaw cycles of the VLPs while maintaining activity. In certain embodiments, the vaccine formulation allows for at least 3 freeze/thaw cycles of the virus particles while maintaining at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% activity. In certain embodiments, the vaccine formulations allow at least 3 freeze/thaw cycles of the virus particles while maintaining at least about 30% activity.

### 4.14. Antibodies Subsequently Generated by Subjects/Patients Administered the Peptide VLP Vaccine Compositions

In one aspect, the disclosure provides a method for generating antibodies against the at least one immunogenic antigen of an infectious agent using the recombinant VLP particles described herein. The generated antibodies may be isolated by standard techniques known in the art (e.g., immunoaffinity chromatography, centrifugation, precipitation, etc.).

Antibodies generated against the at least one immunogenic antigen of an infectious agent by immunization with the recombinant VLP vaccines described herein also have potential uses in diagnostic immunoassays and passive immunotherapy.

Assays in which the antibodies generated by hosts inoculated with the recombinant VLP vaccine described herein can include, but are not limited to, competitive and noncompetitive assay systems using techniques such as radioimmunoassays, ELISA (enzyme-linked immunosorbent assays), “sandwich” immunoassays, precipitin reactions, gel diffusion precipitin reactions, immunodiffusion assays, agglutination assays, complement-fixation assays, immunoradiometric assays, fluorescent immunoassays, protein A immunoassays and immunoelectrophoretic assays, etc.

### 4.15. Kits

Additionally, certain components or embodiments of the compositions can be provided in a kit. For example, the peptide VLP vaccine composition, as well as the related buffers or other components related to administration can be provided in separate containers and packaged as a kit, alone or along with separate containers of any of the other agents from any pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator. In some embodiments, the kits include all components needed for the various stages of treatment. In some embodiments, the compositions may have preservatives or be preservative-free (for example, in a single-use container). In some embodiments, the kit may comprise materials for intramuscular administration. In some embodiments, the kit may comprise buffers, diluents, or emergency allergic reaction treatments, such as epinephriner Benadryl in a separate container.

### 5. EXAMPLES

### Example 1

### 5.1. In-Vivo Study in Mice to Determine Neutralizing Antibody Generation Against SARS-COV-2 using the Spike Protein VLP Vaccine (CB-109)

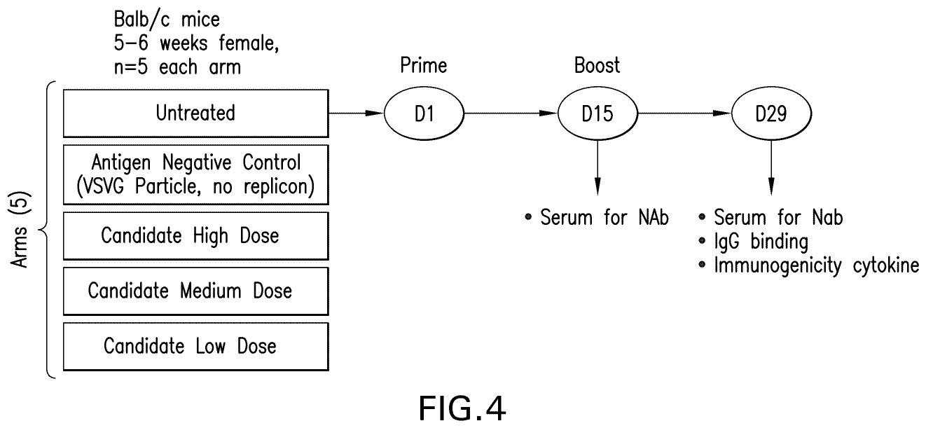

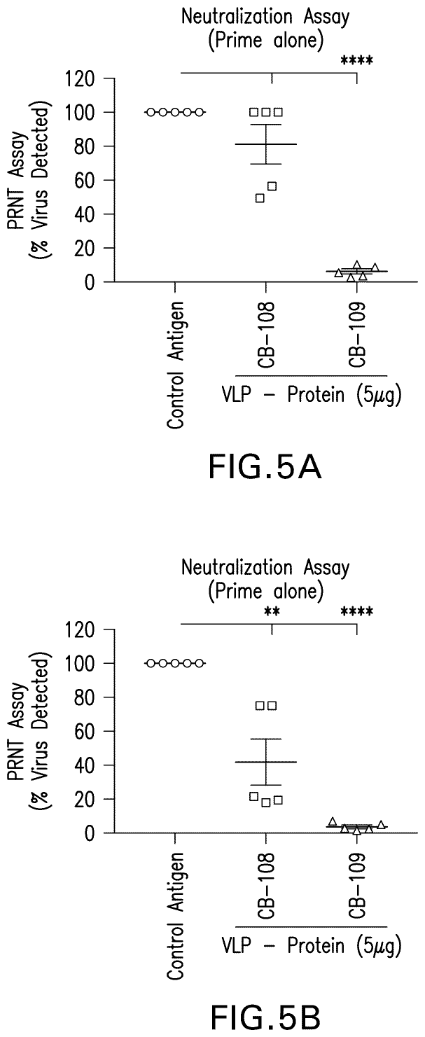

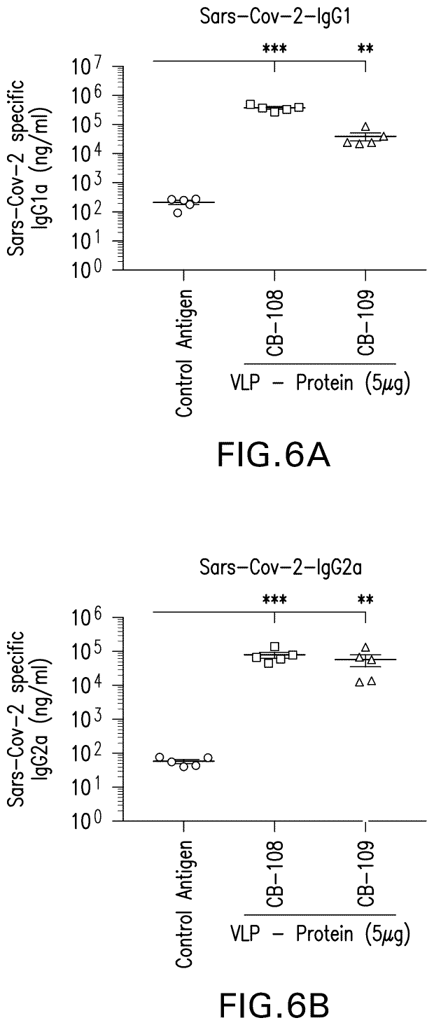

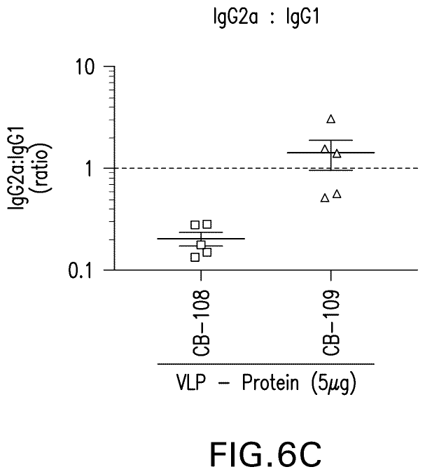

A prime and boost study was designed in 7-week-old female Balb-c mice to test the ability of the vaccines (FIG. 4 peptide VLP candidates CB-108 and CB-109) to generate neutralizing antibodies against SARS-COV-2 (FIGS. 5A-5B). The candidate designs are shown in FIGS. 1A-B, and various sequence information is shown in FIGS. 2A-2F, including the complete spike glycoprotein (S-protein) (SEQ ID NO:2 and amino acid sequences of the ectodomain spike glycoprotein with the hemagglutinin (HA) transmembrane region from influenza virus (SEQ ID NO:4). Mice (n=5 or 7) were injected intramuscularly with 50 μl of either vaccine at Day 1 (Prime) and Day 14 (Boost) in a 4-week study. Serum samples were collected pre-boost and at the end of study to determine neutralizing antibodies and induction of immunoglobulins. Data obtained were compared to a negative antigen control cohort. Dose was calculated based on infectivity assays for VLP-protein. These data are shown in FIGS. 6A-6C. Candidate construct 1 (CB-108) contains the full-length spike protein on the surface of the VLP. Candidate construct 2 (CB-109) contains the ectodomain of SARS-COV-2 spike glycoprotein fused with the HA domain (SEQ ID NO:4) on the surface of the peptide VLP. Candidate 2 has provided optimal observed data and is being termed CB-109.

### 5.2. Western Blot Characterization of Peptide VLP Particle

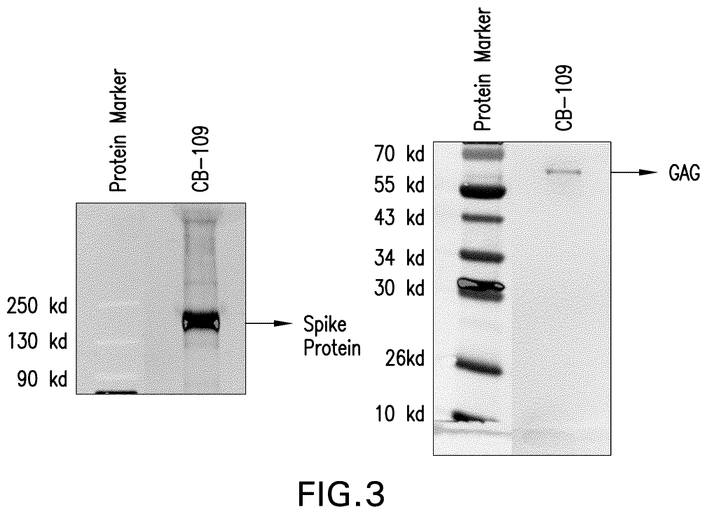

FIG. 3 is a western blot confirming Spike and GAG protein expression in the CB-109 particle.

### 5.3. Antigen Specific IgG Elisa

The antigen-specific IgG, IgG1 and IgG2a titers in mouse sera were assessed by a semi-quantitative E LISA. Protocol used was adapted from Mckay PF et.al, Nature Communication, 2020 (https://doi.org/10.1038/s41467-020-17409-9). Briefly, high binding ELISA plates were coated with 100 μL per well of 1 μg/mL recombinant SARS-COV-2 protein in PBS. For the standard IgG/IgG1/IgG2a, 3 columns on each plate were coated with 1:1000 dilution each of goat anti-mouse Kappa and Lambda light chains. After overnight incubation at 4° C., the plates were washed 4 times with PBS-Tween 20 0.05% (v/v) and blocked for 1 h at 37° C. with 200 μL per well blocking buffer (1% BSA (w/v) in PBS-Tween-20 0.05%(v/v)). The plates were then washed and the diluted samples or a 5-fold dilution series of the standard IgG (or IgG1 or IgG2) added using 50 μL per well volume. Plates were incubated for 1 h at 37° C., then washed and secondary antibody added at 1:2000 dilution in blocking buffer (100 μL per well) using either anti-mouse IgG-HRP, anti-mouse IgG1-HRP or anti-mouse IgG2a-HRP. After incubation and washes, plates were developed using 50 μL per well TMB (3,3′, 5,5′-tetramethylbenzidine) substrate and the reaction stopped after 5 min with 50 μL per well stop solution. The absorbance was read on a Spectrophotometer at 450 nm. These data are shown in FIGS. 6A-6C, which illustrate the Th1/Th2 skew in response to SARS-COV-2 peptide VLP vaccine. IgG1 (FIG. 6A) and IgG2a (FIG. 6B) responses in mice vaccinated with peptide VLP. N=5 animals. The mice demonstrated a Th1 bias (FIG. 6C) Data shown as Mean±SD. Significance compared to antigen control was determined by performing a one-way ANOVA; **p<0.001, ***p<0.0001.

### 5.4. Particle Manufacturing and Concentration

### 5.4.1. SARS-COV-2 VLP-Protein Particle

Two plasmids individually coding for RSV-GAG and Spike protein or ectodomain fused to the hemagglutinin (HA) region, in the ratio of 1:1 or others were electroporated into a production cell line (BHK-21, BHK/AC9, HEK293, VERO, A549 among others) and the cells incubated for 48-96 hours. In addition to electroporation, PEI (polyethylenimine) based transfection was also used. In this case, different ratios of PEI:DNA were used including 1:1, 1:2, 1:3. After incubation, the cells and media was collected and spun down at 1000×g for 10 min. The purified supernatant was then subjected to ultracentrifugation (134000×g for 4 hrs) in 20% glycerol. The pellet formed was dissolved in 1 ×PBS and stored at −80 C. In some instances, the supernatant was subjected to anion exchange resins, purified using HPLC and TFF prior to concentration and storage at −80 C.

### 5.5. Temperature Stability Experiments

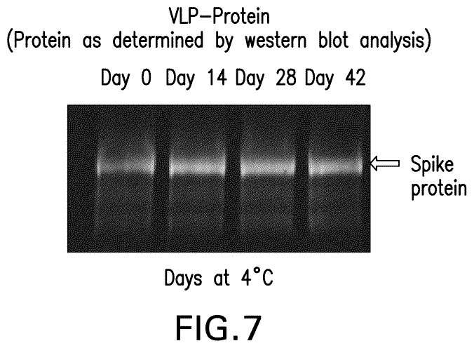

The purified and concentrated peptide VLP vaccines were stored at 4° C. Aliquots were collected at different times and measured for protein expression (peptide VLP). Stability of VLP-protein was determined by western blot analysis or SARS-COV-2 Spike specific ELISA. Stability has been demonstrated at from about 2° C.-8° C. for at least one-six months; and at about −80° C. for at least about 2 years. FIG. 7 shows the stability measured by infectivity assay for up to twelve weeks.

### 5.6. SARS-COV-2 Spike Protein VLP Vaccine (CB-109) Safety/Toxicity Studies in Rabbits

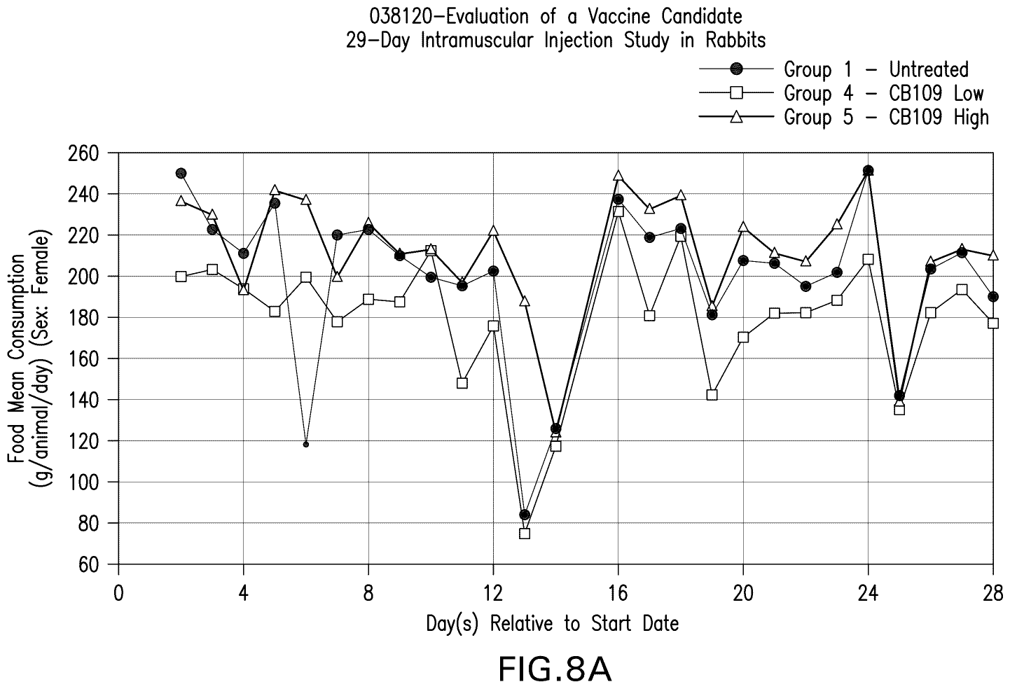

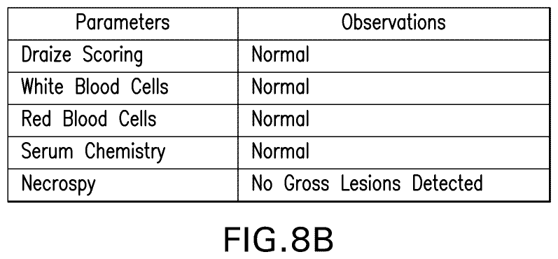

Two doses (VLP—150 μg and 15 μg) of each of the vaccine candidates were each administered, on Days 1 and 15 via intramuscular injection (0.5 mL) in the right hindlimb, to different groups of two male and female New Zealand White (NZW) rabbits. Dose selected was the human equivalent dose calculated from the mice studies. The control NZW rabbits (2/sex) were untreated. All rabbits were terminated on Day 29. Animals were monitored for clinical observations twice a day, predose detailed clinical observations, dermal observations (Draize scores) at the injection site prior to and for 3 days following each dose, weekly body weights, daily quantitatively assessment of food consumption, body temperature prior to each dose and daily thereafter until the temperature of all animals was within the normal range (38-39.9° C.), and clinical pathology (hematology and serum chemistry parameters) on Days 15 and 29. Postmortem assessment included gross necropsy, measurement of selected organ weights (heart, kidneys, liver, lung, spleen, and thymus), and microscopic evaluation of a selected tissues (brain, gallbladder, heart, injection site, kidneys, liver, lungs, ovaries, spleen, testes, and thymus). Samples for immunogenicity tests (Sars-Cov-2 neutralizing antibody and Sars-Cov-2 specific IgG) were collected from all animals on Day 1 (predose), Day 15 (predose), and Day 29. These data are shown in FIGS. 8A-8B, where there was no impact on body weight gain, food consumption (FIG. 8A) and other parameters measured (FIG. 8B).

### Example 2

### 5.7. Clinical Phase-1 Design for SARS-COV-2 Spike Protein VLP Vaccine (CB-109)

The Phase-1 study will evaluate the safety, reactogenicity and immunogenicity of CB-109 in a dose-ranging study. Three dose levels of CB-109 will be given to healthy adults in two doses, 28 days apart, and three dose levels of CB-109 administered as single dose to healthy adults. Assessments will be conducted in a seven-Arm study, n=175 volunteer, phase I, open-label, dose-ranging study involving men and women ages 18 and up who meet all eligibility criteria. Dose range will be evaluated in a Prime alone (single dose) and a Prime and Boost (2 dose) regimen. Dose will be selected on Human Dose Equivalent calculations based on concentrations in mice and for the protein vaccine is expected to be 1 nanogram (0.001 microgram) to 100 micrograms Spike protein or total protein. Each dose will be administered as 0.3mL-0.5 mL vaccine via intra-muscular injection on Day 1 for prime cohort and Days 1 and 29 for the booster cohort. Subjects will be monitored up to twelve months post-vaccination (Day 394), including visits one, two, and four weeks after each vaccination, and three and six months after the second vaccination. Primary and Secondary outcomes measured up to Day 57 (28 days after the booster dose on Day 29) may be used to generate preliminary critical data package to secure Fast Track designation and approval to recruit to Phase-II volunteer study.

Experimental Arm One: CB-109 Dose Level 1: A single value within the range of 0.001 microgram to twenty (20) micrograms of protein will be tested.
Participants will receive two intramuscular (IM) injections of CB-109 at Dose Level 1, the first dose on Day 1, and the second dose on Day 29.

Experimental Arm Two: CB-109 Dose Level 2: A single value within the range of twenty (20) micrograms to sixty (60) micrograms of protein will be tested.
Participants will receive two IM injections of CB-109 at Dose Level 2, the first dose on Day 1 and the second dose on Day 29.

Experimental Arm Three: CB-109 Dose Level 3: A single value within the range of sixty (60) micrograms to one hundred (100) micrograms of protein will be tested.
Participants will receive two IM injections of CB-109 at Dose Level 3, the first dose on Day 1, and the second dose on Day 29.

Experimental Arm Four: CB-109 Dose Level 1: A single value within the range of 0.001 microgram to twenty (20) micrograms of protein will be tested.
Participants will receive one IM injection of CB-109 at Dose Level 1 on Day 1 and 1 IM injection of study drug-matching placebo on Day 29.

Experimental Arm Five: CB-109 Dose Level 2: A single value within the range of twenty (20) micrograms to sixty (60) micrograms of protein will be tested.
Participants will receive one IM injection of CB-109 at Dose Level 2 on Day 1 and 1 IM injection of study drug-matching placebo on Day 29.

Experimental Arm Six: CB-109 Dose Level 3: A single value within the range of sixty (60) micrograms to One hundred (100) micrograms of protein will be tested.
Participants will receive one IM injection of CB-109 at Dose Level 3 on Day 1 and 1 IM injection of study drug-matching placebo on Day 29.

Experimental Arm Seven: Study-Drug Matched Placebo
Participants will receive two intramuscular (IM) injections of Study-Drug matched placebo, the first on Day 1, and the second on Day 29.

Primary Outcome measures will include:
1. Number of participants with solicited local and systemic reactogenicity adverse reactions (ARs.) This will be monitored up to Day 36 in the case of prime and boost study arms (7 days after the booster dose on Day 29) and up to Day 7 after the prime dose in case of Prime only arms.
2. Number of participants with unsolicited adverse events (AEs.) This will be monitored up to Day 57 in the case of prime and boost study arms (28 days after the booster dose on Day 29) and up to Day 28 after the prime dose in case of Prime only arms.
3. Number of participants with medically attended AEs (AAAEs), AE of Special Interest (AESIs) and Serious Adverse Events (SAEs). This will be monitored up to Day 394 after the second dose in the case of prime and boost study arms.
4. It is expected that only Grade 1 or Grade 2 reactions will be observed following prime and/or boost administrations, and no serious adverse reactions, such as AAAEs, AESIs, or SAEs are expected in view of the current animal data (See, FIG. 8A-8B).

Secondary Outcome measures will include:
1. Geometric Mean (GM) of SARS-COV2 specific neutralizing antibodies (nAb) on Day 1, Day 29, Day 36, Day 57, Day 209 and Day 394.
2. GM of SARS-COV-2 specific binding antibody (bAb) on Day 1, Day 29, Day 36, Day 57, Day 209 and Day 394.
3. Seroconversion as measured by an increase of SARS-COV-2 specific nAb titer or bAb titer on Day 1, Day 7, Day 29, Day 36, Day 209 and Day 394.

Key Inclusion Criteria for each Subject/Patient:
Understands and agrees to comply with the study procedures and provides written informed consent.
According to the assessment of the investigator, is in good general health and can comply with study procedures.
Body mass index (BMI) of 18 kilograms/square meter (kg/m\hat{ }2) to 35 kg/m\hat{ }2 (inclusive) at the Screening Visit (Day 0).
For female participants of childbearing potential: negative pregnancy test, adequate contraception or has abstained from all activities that could result in pregnancy for at least 28 days prior to the first injection, agreement to continue adequate contraception or abstinence through 3 months following the second injection, and not currently breastfeeding.

Key Exclusion Criteria for each Subject/Patient:
Known history of SARS-COV-2 infection or known exposure to someone with SARS-CoV-2 infection or COVID-19 in the past 30 days.
Positive serology results for SARS-COV-2 at the Screening Visit. A negative serological test for SARS-COV-2, performed on a blood sample obtained at the Screening Visit, is required before a participant can be dosed.
Travel outside of the region of participation in the 28 days prior to the Screening Visit (Day 0).
Prior administration of an investigational, authorized, or licensed CoV (for example, SARS-COV-2, SARS-COV, or Middle East Respiratory Syndrome [MERS]-COV) vaccine, based on medical history interview.
Current treatment with investigational agents for prophylaxis against COVID-19.
Recent (within the last 12 months) use of a dermal filler.
Has a medical, psychiatric, or occupational condition that may pose additional risk as a result of participation or that could interfere with safety assessments or interpretation of results according to the investigator's judgment.
Has received systemic immunosuppressants or immune-modifying drugs for >14 days in total within 6 months prior to Screening (for corticosteroids, 10 milligrams (mg)/day of prednisone equivalent) or is anticipating the need for immunosuppressive treatment at any time during participation in the study.
Has received or plans to receive any licensed vaccine 28 days prior to the first injection (Day 1) or plans to receive a licensed vaccine within 28 days before or after any study injection, with the exception of licensed influenza vaccines, which may be received more than 14 days before the first study injection or more than 14 days after the second study injection.
Receipt of systemic immunoglobulins or blood products within 3 months prior to the Screening Visit (Day 0) or plans for receipt during the study.
Current use of any inhaled substance (for example, tobacco or cannabis smoke, nicotine vapors).
History of chronic smoking (1 cigarette a day) within 1 year of the Screening Visit.
Resides in a nursing home.
Has donated 450 milliliters (mL) of blood products within 28 days prior to the Screening Visit or plans to donate blood products during the study.

Participated in an interventional clinical study within 28 days prior to the Screening Visit based on the medical history interview or plans to do so while participating in this study.

SEQUENCE DESCRIPTIONS

SEQ ID NO: 1
Nucleotide sequence encoding Coronavirus full length spike protein
atgtttgtgttcctggtgcttctgccacttgtttcctcacaatgcgtcaatctcacaacaagaacgcagcttccgcctgcatatacaaa
tagtttcaccagaggcgtttattatccagataaggtctttcgaagctctgtcctccattctacgcaagatctgttcttgccattcttttcaa
acgtgacgtggtttcacgctattcacgtttctggcactaatggcacgaagcgctttgacaaccccgtccttccgttcaacgatggcg
tctatttcgcgagtacagagaagtcaaatataatccgcggctggatatttggcacaaccctcgatagtaaaacacaatcccttctga
tcgtaaataacgcaacgaatgtggtgattaaggtatgcgagtttcaattttgcaacgacccgttccttggcgtttattatcacaaaaat
aacaagtcctggatggagtccgaattcagagtgtattcttctgccaacaattgcacctttgagtacgtgtcccagcccttcttgatgg
atctggaaggaaaacagggcaatttcaagaatttgagagagttcgtcttcaaaaacatcgatggttattttaagatctatagcaagc
acaccccgataaacctcgtacgggaccttccccaggggtttagcgcactggagcctcttgtagatctcccgattggcatcaatata
accaggtttcaaactcttcttgcacttcaccgaagctatcttacgcccggcgatagctcttccggttggacagccggcgccgcggc
ctactatgtgggatatctccagcctagaacctttctgcttaagtacaatgaaaacggcactattacagacgccgtagattgcgctctg
gatcctcttagcgaaactaagtgtactctgaaatcttttacggtagagaagggaatttatcaaacaagtaatttccgggtacaaccca
cagagagcatcgtccggtttccgaacattacgaatctttgcccgtttggcgaagtcttcaatgcaacacgcttcgcttccgtttatgc
gtggaatcgcaagaggatctctaactgcgttgctgactacagtgttctttataacagcgctagcttttctacattcaaatgttacgggg
taagccccaccaagctcaacgacttgtgttttaccaatgtttatgcggattctttcgtgattcgcggtgatgaagttaggcagattgcg
ccaggacagactgggaagattgccgactataactataagctccctgacgacttcactgggtgtgtcattgcttggaacagcaaca
atctcgacagcaaagttggaggcaactataattatctctaccggctttttcgcaagtctaacctcaagcccttcgaaagagacatca
gtactgagatctatcaagcaggaagcacgccatgcaacggtgtcgagggcttcaactgttattttcctcttcaatcctacggctttca
accgacaaacggcgtcggatatcagccttatcgagtcgttgtactttctttcgaacttctgcacgcaccagctactgtctgcgggcc
taaaaaatctacaaacctggtgaaaaataaatgcgttaattttaattttaacggtttgaccggcaccggggtccttactgagtcaaata
aaaagttcctgccgtttcagcagtttggccgcgatattgccgatactacagacgcggtgcgggaccctcaaactctcgaaatcctg
gatataacgccttgctcttttggcggggtaagcgtcattaccccaggtacaaatacgtcaaatcaggtcgctgttctgtaccaagac
gtcaattgcaccgaggtcccagtagctatccatgccgatcaactcacgccaacctgggggtatactctacgggatccaacgtctt
ccaaaccagggcggggtgcctcatcggagccgaacacgtaaacaatagctatgagtgcgatattcccataggggccggaatct
gcgcttcataccaaacccagactaattctccccgacgagcccgaagcgtggcatctcaaagcataatagcgtacacgatgagctt
gggagcagaaaactcagtggcgtatagcaataactccatcgcgataccaaccaattttacaatcagtgtgactacagagatcttgc
cggttagtatgacaaagacttcagtggattgcacgatgtacatctgtggagatagtaccgagtgcagcaaccttttgttgcagtatg
gctcattttgtacccaactcaatcgggccctgaccggtattgcagttgaacaggataaaaacacacaagaggtctttgcacaagtt
aaacaaatatacaagaccccgcctatcaaagactttggtggttttaattttagccaaattcttcccgacccgtcaaagccgagtaag
cggtcattcatagaggatctcctctttaataaggtaaccttggctgacgcgggatttattaaacagtacggtgactgtttgggagata
ttgcagctcgagacttgatctgcgcccagaaattcaacggtctgactgttttgcctccgctcttgacggacgagatgatcgcccagt
atacttccgccttgctcgctggtacgatcacctccggctggacctttggcgctggagctgctctccagatcccattcgcgatgcag
atggcttaccgatttaacggtattggcgttacacaaaatgtattgtacgagaaccaaaaactcatcgctaatcaattcaactcagcta
ttggcaaaattcaagactcattgtcctctacagccagcgccttgggtaaattgcaagacgtagtgaaccaaaatgcacaggccttg
aacactctggtcaagcaactcagttccaattttggtgcaatctcatccgttctcaacgatatactgtctcgcctggataaggtggaag
ctgaggtgcaaatcgacagactcataacgggaaggctgcagagtctgcagacctatgtcactcagcagcttattcgagcggccg
aaatccgcgcttccgccaatttggctgctactaaaatgagcgaatgcgtactcgggcaatctaaacgagtcgacttttgtggcaaa
ggctaccacctcatgtcatttccccagagtgcgccacacggggtggttttccttcatgtcacctacgtgcccgcccaagaaaagaa
ctttactaccgcaccggccatctgccatgatggaaaggcccatttcccgagagagggcgtatttgtctcaaacggtactcactggt
ttgtgacacaacgcaatttctacgaaccccaaattataaccactgacaacacatttgtctctggaaactgtgatgtcgttattggtata
gttaacaacacggtctacgacccactccagccagagctggactcttttaaagaagaactcgacaagtattttaaaaatcacacgag
tcccgacgttgaccttggagacattagtgggatcaacgcgagtgtcgttaacatccaaaaggagattgatcgcctcaatgaggtg
gccaagaatctcaacgaatccctcatagatctccaagagctcggtaagtatgagcagtatattaagtggccctggtatatatggctc
ggctttatcgccggcctgattgcaatagttatggttacgataatgctgtgctgtatgacttcatgctgttcttgtttgaaaggctgttgct
catgtgggagttgttgcaagtttgatgaggacgacagtgaacccgtattgaagggtgtgaagcttcattacacttaa
SEQ ID NO: 2
AA sequence Coronavirus full length spike protein
S1 domain is underlined
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNERVQPTESIVRFPNITNLCPFGEVENATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT
SEQ ID NO: 3
Nucleotide sequence (codon optimized) encoding the ectodomain COVID-19 spike
glycoprotein (S-protein) with C-terminus of the hemagglutinin (HA) transmembrane
region from Influenza, linker sequence and cytoplasmic tail
atgtttgtgttcctggtgcttctgccacttgtttcctcacaatgcgtcaatctcacaacaagaacgcagcttccgcctgcatatacaaatagt
ttcaccagaggcgtttattatccagataaggtctttcgaagctctgtcctccattctacgcaagatctgttcttgccattcttttcaaacgtgac
gtggtttcacgctattcacgtttctggcactaatggcacgaagcgctttgacaaccccgtccttccgttcaacgatggcgtctatttcgcga
gtacagagaagtcaaatataatccgcggctggatatttggcacaaccctcgatagtaaaacacaatcccttctgatcgtaaataacgcaa
cgaatgtggtgattaaggtatgcgagtttcaattttgcaacgacccgttccttggcgtttattatcacaaaaataacaagtcctggatggag
tccgaattcagagtgtattcttctgccaacaattgcacctttgagtacgtgtcccagcccttcttgatggatctggaaggaaaacagggca
atttcaagaatttgagagagttcgtcttcaaaaacatcgatggttattttaagatctatagcaagcacaccccgataaacctcgtacggga
ccttccccaggggtttagcgcactggagcctcttgtagatctcccgattggcatcaatataaccaggtttcaaactcttcttgcacttcacc
gaagctatcttacgcccggcgatagctcttccggttggacagccggcgccgcggcctactatgtgggatatctccagcctagaaccttt
ctgcttaagtacaatgaaaacggcactattacagacgccgtagattgcgctctggatcctcttagcgaaactaagtgtactctgaaatcttt
tacggtagagaagggaatttatcaaacaagtaatttccgggtacaacccacagagagcatcgtccggtttccgaacattacgaatctttg
cccgtttggcgaagtcttcaatgcaacacgcttcgcttccgtttatgcgtggaatcgcaagaggatctctaactgcgttgctgactacagt
gttctttataacagcgctagcttttctacattcaaatgttacggggtaagccccaccaagctcaacgacttgtgttttaccaatgtttatgcgg
attctttcgtgattcgcggtgatgaagttaggcagattgcgccaggacagactgggaagattgccgactataactataagctccctgacg
acttcactgggtgtgtcattgcttggaacagcaacaatctcgacagcaaagttggaggcaactataattatctctaccggctttttcgcaa
gtctaacctcaagcccttcgaaagagacatcagtactgagatctatcaagcaggaagcacgccatgcaacggtgtcgagggcttcaac
tgttattttcctcttcaatcctacggctttcaaccgacaaacggcgtcggatatcagccttatcgagtcgttgtactttctttcgaacttctgca
cgcaccagctactgtctgcgggcctaaaaaatctacaaacctggtgaaaaataaatgcgttaattttaattttaacggtttgaccggcacc
ggggtccttactgagtcaaataaaaagttcctgccgtttcagcagtttggccgcgatattgccgatactacagacgcggtgcgggaccc
tcaaactctcgaaatcctggatataacgccttgctcttttggcggggtaagcgtcattaccccaggtacaaatacgtcaaatcaggtcgct
gttctgtaccaagacgtcaattgcaccgaggtcccagtagctatccatgccgatcaactcacgccaacctggcgggtatactctacggg
atccaacgtcttccaaaccagggcggggtgcctcatcggagccgaacacgtaaacaatagctatgagtgcgatattcccataggggc
cggaatctgcgcttcataccaaacccagactaattctccccgacgagcccgaagcgtggcatctcaaagcataatagcgtacacgatg
agcttgggagcagaaaactcagtggcgtatagcaataactccatcgcgataccaaccaattttacaatcagtgtgactacagagatcttg
ccggttagtatgacaaagacttcagtggattgcacgatgtacatctgtggagatagtaccgagtgcagcaaccttttgttgcagtatggct
cattttgtacccaactcaatcgggccctgaccggtattgcagttgaacaggataaaaacacacaagaggtctttgcacaagttaaacaaa
tatacaagaccccgcctatcaaagactttggtggttttaattttagccaaattcttcccgacccgtcaaagccgagtaagcggtcattcata
gaggatctcctctttaataaggtaaccttggctgacgcgggatttattaaacagtacggtgactgtttgggagatattgcagctcgagactt
gatctgcgcccagaaattcaacggtctgactgttttgcctccgctcttgacggacgagatgatcgcccagtatacttccgccttgctcgct
ggtacgatcacctccggctggacctttggcgctggagctgctctccagatcccattcgcgatgcagatggcttaccgatttaacggtatt
ggcgttacacaaaatgtattgtacgagaaccaaaaactcatcgctaatcaattcaactcagctattggcaaaattcaagactcattgtcctc
tacagccagcgccttgggtaaattgcaagacgtagtgaaccaaaatgcacaggccttgaacactctggtcaagcaactcagttccaatt
ttggtgcaatctcatccgttctcaacgatatactgtctcgcctggataaggtggaagctgaggtgcaaatcgacagactcataacgggaa
ggctgcagagtctgcagacctatgtcactcagcagcttattcgagcggccgaaatccgcgcttccgccaatttggctgctactaaaatg
agcgaatgcgtactcgggcaatctaaacgagtcgacttttgtggcaaaggctaccacctcatgtcatttccccagagtgcgccacacgg
ggtggttttccttcatgtcacctacgtgcccgcccaagaaaagaactttactaccgcaccggccatctgccatgatggaaaggcccattt
cccgagagagggcgtatttgtctcaaacggtactcactggtttgtgacacaacgcaatttctacgaaccccaaattataaccactgacaa
cacatttgtctctggaaactgtgatgtcgttattggtatagttaacaacacggtctacgacccactccagccagagctggactcttttaaag
aagaactcgacaagtattttaaaaatcacacgagtcccgacgttgaccttggagacattagtgggatcaacgcgagtgtcgttaacatcc
aaaaggagattgatcgcctcaatgaggtggccaagaatctcaacgaatccctcatagatctccaagagctcggtaagtatgagcagta
a
SEQ ID NO: 4
Amino acid sequence of the ectodomain COVID-19 spike glycoprotein (S-protein) with
hemagglutinin (HA) transmembrane region from Influenza, linker domain and
cytoplasmic tail from HA.
S1 domain is underlined
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNERVQPTESIVRFPNITNLCPFGEVENATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQG
TYDYPKYSEESKLNREKIDGVKLESMGVYQILAIYSTVASSLVLLVSLGAISFWMCSN
GSLQCRICI
Signal Domain (SEQ ID NO: 5)
MFVFLVLLPLVSS
N-terminus Domain (SEQ ID NO: 6)
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNF
Receptor Binding Domain (SEQ ID NO: 7)
RVQPTESIVRFPNITNLCPFGEVENATRFASVYAWNRKRISNCVADYSVLYNSASF
STFKCYGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTPCNGVE
GFNCYFPLQSYGFQPTNGVGYQPYRV
S2 Domain (SEQ ID NO: 8)
VVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVP
VAIHADQLTPTWRVYSTGSNVFQTRAGCLIGAEHVNNSYECDIPIGAGICASYQT
QTNSPRRARSVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFAQVKQIY
KTPPIKDFGGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQ
LIRAAEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASV
VNIQKEIDRLNEVAKNLNESLIDLQELGKYEQ
Linker Domain (SEQ ID NO: 9)
GTYDYPKYSEESKLNREKIDGVKLESMGVY
Transmembrane Domain from HA (SEQ ID NO: 10)
QILAIYSTVASSLVLLVSLGAISFWM
Cytoplasmic Tail (SEQ ID NO: 11)
CSNGSLQCRICI

### 6. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A virus like particle (VLP), the VLP comprising:
a. a capsid protein comprising a retroviral gag protein, and
b. an envelope protein, wherein the envelope protein comprises at least one heterologous polypeptide or fragment thereof, wherein the VLP does not contain alphavirus genetic material.

2. The VLP of claim 1, wherein the at least one heterologous polypeptide or fragment thereof is a cell surface protein, receptor, or an antigen binding protein.

3. The VLP of claim 2, wherein the at least one heterologous polypeptide or fragment thereof is immunogenic.

4. The VLP of claim 3, wherein the at least one heterologous polypeptide or fragment thereof exhibits at least 80% identity to at least one immunogenic antigen of an infectious agent.

5. The VLP of claim 4, wherein the infectious agent is a bacterium. The VLP of claim 4, wherein the infectious agent is a virus.

7. The VLP of claim 6, wherein the virus is a coronavirus.

8. The VLP of claim 7, wherein the coronavirus is SARS-COV-2.

9. The VLP of any of the previous claims, wherein heterologous polypeptide or fragment thereof has 80% identity to a coronavirus spike protein, a membrane protein, a hemagglutinin esterase (HE), a hemagglutinin from influenza virus, or an envelope protein, or any fragment or combination thereof.

10. The VLP of claim 9, wherein heterologous polypeptide or fragment thereof has 80% identity to a coronavirus spike protein or a chimeric protein comprising the SARS-COV-2 ectodomain spike glycoprotein (S-protein) and further comprising the hemagglutinin (HA) transmembrane region from Influenza virus.

11. The VLP of claim 10, wherein the spike protein sequence shares at least 90% identity with a coronavirus spike protein of SEQ ID NO:2 or ectodomain amino acids 1-1208 of SEQ ID NO: 2, or a protein comprising the SARS-COV-2 ectodomain spike glycoprotein (S-protein) and hemagglutinin (HA) transmembrane region from Influenza virus (SEQ ID NO:4) or any combination thereof.

12. The VLP of claim 11, wherein the spike protein sequence comprises SEQ ID NO: 2 or the ectodomain region of amino acids 1-1208 of SEQ ID NO: 2, or chimeric protein of the SARS-COV-2 ectodomain spike glycoprotein (S-protein), a linker fragment, a hemagglutinin (HA) transmembrane region from Influenza virus, a cytoplasmic tail of the Influenza hemagglutinin (SEQ ID NO:4-11), or any combination thereof.

13. The VLP of claim 9 or 10, wherein the spike protein sequence is encoded by a codon optimized sequence of SEQ ID NO: 1; or a codon optimized ectodomain sequence of amino acids 1-1208 of SEQ ID NO: 1, or a codon optimized sequence of SEQ ID NO:3 encoding the ectodomain COVID-19 spike glycoprotein (S-protein) with hemagglutinin (HA) transmembrane region from Influenza.

14. The VLP of any of the previous claims, wherein the VLP does not comprise or express a retroviral pol gene.

15. The VLP of any of the previous claims, wherein the retroviral gag protein is encoded by a polynucleotide sequence derived from Rous sarcoma virus (RSV).

16. The VLP of any of the previous claims, wherein the envelope further comprises a transmembrane region to anchor the at least one heterologous polypeptide or fragment thereof to the capsid protein, wherein the transmembrane region is a glycoprotein, or fragment or derivative thereof.

17. The VLP of any of the previous claims, wherein the at least one heterologous polypeptide or fragment of the envelope is anchored to the capsid protein by a transmembrane domain from VSV-G or from any RSV.

18. The VLP of claim 17, wherein the target immune cell is a dendritic cell.

19. The VLP of any of the previous claims, wherein the VLP is capable of presenting the immunogenic antigen to a target cell in a subject which induces a cellular and/or humoral immune response to SARS-COV-2 in the subject.

20. The VLP of claim 19, wherein the immune response including a T cell response is induced by a dosing regimen comprising one or two administrations of the VLP, wherein the dose is sufficient to induce an immune response against coronavirus in a subject.

21. The VLP of claim 20, wherein the dosing regimen comprises one administration of the VLP, wherein the one dose administration is sufficient to induce an immune response against the coronavirus in a subject.

22. A pharmaceutical composition comprising the VLP of any of the previous claims.

23. The pharmaceutical composition of claim 22, wherein the composition comprises, optionally, a pharmaceutically acceptable carrier, diluent, adjuvant and/or additive, or any combination thereof.

24. The pharmaceutical composition of claim 22 or 23, which is capable of inducing an immune response against coronavirus in a subject.

25. The pharmaceutical composition of any of claims 22-24, wherein following administration of the composition to the subject, the VLP is capable of inducing a T cell response against coronavirus.

26. The pharmaceutical composition of claim 22, wherein the coronavirus is SARS-COV-2 (COVID-19).

27. The VLP or pharmaceutical of any of claims 1-26, for use in diminishing or preventing a coronavirus infection in a mammalian subject.

28. The VLP or pharmaceutical composition of claim 27, wherein said diminishing or preventing comprises inducing coronavirus-specific immunity against SARS-COV-2 (COVID-19).

29. The VLP or pharmaceutical composition of any of claims 1-28 for use in inducing cellular and or humoral immunity in a mammalian subject.

30. The VLP or pharmaceutical composition of any of claims 1-29 for use in inducing or eliciting an immune response in a mammalian subject.

31. The VLP or pharmaceutical composition of claim 30, wherein said inducing or eliciting an immune response is an immune response against SARS-COV-2 (COVID-19).

32. The VLP or pharmaceutical composition of claim 31, for use in inducing neutralizing antibodies against SARS-COV-2 in a mammalian subject.

33. A method of diminishing or preventing a coronavirus infection in a mammalian subject comprising administering the VLP or pharmaceutical composition of any of claims 1-32 to the subject.

34. A method of inducing cellular and or humoral immunity against a coronavirus in a mammalian subject, comprising administering the VLP or pharmaceutical composition of any of claims 1-32 to the subject.

35. A method of eliciting an immune response against a coronavirus in a mammalian subject, comprising administering the VLP or pharmaceutical composition of any of claims 1-32 to the subject.

36. A method of inducing neutralizing antibodies against SARS-COV-2 in a subject, comprising administering the VLP or pharmaceutical composition of any of claims 1-32 to the subject.

37. The method of any one of claims 33-36, wherein the method further includes inducing a T cell response against the coronavirus.

38. The method of claim 37, wherein the said T cell response is induced by a regimen comprising one or at least two administrations.

39. The method of any one of claims 33-38, wherein the immune response is induced by a regimen comprising one administration of the VLP or pharmaceutical composition.

40. The method of any one of claims 33-39, wherein the coronavirus is SARS-COV-2.

41. The method of claim 40, wherein the VLP is capable of delivering the immunogenic antigen to a target cell in a subject, after which the subject is capable of mounting a cellular and/or humoral immune response to SARS-COV-2 in the subject.

42. A method of producing the VLP of any of claims 1-21, comprising:
co-transforming a eukaryotic cell with:
i. a first plasmid or vector comprising a polynucleotide encoding the retroviral gag protein; and
ii. a second plasmid or vector comprising a polynucleotide encoding a at least one heterologous polypeptide or fragment thereof or one or more polypeptide or fragment thereof which exhibits at least 80% identity to at least one immunogenic antigen of an infectious agent;
iii. culturing the co-transformed eukaryotic cell under conditions suitable to cause each vector to produce its encoded product, thereby producing the VLP; and
iv. isolating the VLP from the eukaryotic cell.

43. A VLP produced by the method of claim 42.

44. A method of diminishing or preventing a coronavirus infection in a mammalian subject comprising administering the VLP of claim 43, to a subject.

45. A method of inducing cellular and or humoral immunity in a mammalian subject, comprising administering the VLP of claim 43, to a subject.

46. A method of eliciting an immune response in a mammalian subject, comprising administering the VLP of claim 43, to a mammalian subject.

47. A method of inducing neutralizing antibodies against SARS-COV-2 in a mammalian subject, comprising administering the VLP of claim 43, to a subject.

48. The method of any one of claims 44-47, wherein the method further includes inducing a T cell response against the coronavirus.

49. The method of claim 48, wherein the said T cell response is induced by a regimen comprising at least one or at least two administrations.

50. The method of claim 49, wherein the coronavirus is SARS-COV-2 (COVID-19).

51. A method of claim 50, wherein the VLP is capable of delivering the immunogenic antigen to a target cell in the subject, after which the subject is capable of mounting a cellular and/or humoral immune response to SARS-COV-2 in the subject.

52. The VLP or composition of any of claims 1-32, wherein the VLP is stable at from the range of about 4° C.-10° C. for at least about one-six months.

53. The VLP or composition of claim 52, wherein the VLP is stable for at least about six to nine months.

54. The VLP or composition of claim 53, wherein the VLP is stable for at least about nine to twelve months.

55. The VLP or composition of any of claims 1-32, wherein the VLP is stable at about −80° C. for at least about one year.

56. The VLP or composition of claim 55, wherein the VLP is stable for about two years.

57. The VLP or composition of claim 56, wherein the VLP is stable for about three years.

58. The method of any of claim 33-41 or 44-51, wherein an effective dose of the VLP for inducing efficacious immunity is equivalent to about 1 μg-1000 μg of total protein.

59. The method of claim 58, wherein the effective dose of the VLP is equivalent to at least about 1 μg-100 μg.

60. The VLP or composition of any of claims 1-32, wherein the VLP is capable of delivering one or more immunogenic antigens to a target cell in a subject exposing the subject to the antigen, after which exposure the subject is capable of producing a cellular and/or humoral immune response to Severe Acute Respiratory Syndrome (SARS-COV), SARS-COV-2, and/or variants of SARS-COV-2.
